(19) United States
(12) Patent Application Publication
(10) Pub. No.: US 2005/0026279 A1
(43) Pub. Date: Feb. 3, 2005

(54) Surgical grafts and methods of preparation

(76) Inventors: Scheffer C.G. Tseng, Pinecrest, FL (US); Edgar M. Espana, Miami, FL (US)

Correspondence Address: ARENDT & ASSOCIATES INTELLECTUAL PROPERTY GROUP, 1740 MASSACHUSETTS AVENUE, BOXBOROUGH, MA, 01719-2209, US

(21) Appl. No.: 10/833,502

(22) Filed: Apr. 28, 2004

Related U.S. Application Data
(60) Provisional application No. 60/465,989, filed on Apr. 28, 2003.
(60) Provisional application No. 60/473,007, filed on May 22, 2003.

(51) Int. Cl. C12N 5/00; C12N 5/02

(52) U.S. Cl. 435/378; 435/183

(57) ABSTRACT

The present invention relates to the discovery of a method of enzymatically, consistently and reproducibly, isolating a viable, intact limbal epithelial sheet that retains stem cell characteristics in the basal epithelium. The method comprises the steps of: a) obtaining limbus from a biopsy of a donor eye of a living individual, a living-related individual, or from a cadaveric eye, the limbus comprising limbal epithelium and an underlying stroma; b) contacting the limbus with a solution comprising Dispase 2, for a period of time and under conditions sufficient to loosen a limbal epithelial sheet from the stroma, thereby forming a loosely adherent limbal epithelial sheet; and c) mechanically separating the loose epithelial sheet from the underlying stroma, thereby isolating a substantially intact, viable, limbal epithelial sheet. Also disclosed are a new culture system to achieve ex vivo expansion of human corneal keratocytes while maintaining their characteristic dendritic morphology and continuous expression of keratocan even in the presence of high concentrations of serum by growing them on the stromal matrix of the human amniotic membrane (AM), and a surgical graft comprising keratocytes on amniotic membrane.

### RELATED APPLICATIONS

This application claims the benefit of U.S. Provisional Application No. 60/465,989, filed on Apr. 28, 2003, and U.S. Provisional Application No. 60/473,007, filed on May 22, 2003, the teachings of both of which are incorporated herein by reference in their entirety.

### GOVERNMENT SUPPORT

The invention described herein was supported, in whole or in part, by research grant number EY 06819 from Department Of Health And Human Services, National Eye Institute, National Institute Of Health, Bethesda, Md. The Government has certain rights in the invention.

### BACKGROUND OF THE INVENTION

Limbal Tissue and the Cornea:

The maintenance of a healthy corneal epithelium under both normal and stressed conditions is achieved by a unique population of stem cells (SC) located in the limbal basal epithelium. See Schermer A, Galvin S, Sun T-T., “Differentiation-related expression of a major 64K corneal keratin in vivo and in culture suggests limbal location of corneal epithelial stem cells,” J Cell Biol. 1986; 103:49-62. “Epithelial neoplasias,” diseases that affect SC, frequently involve the limbal area. Destruction of the limbal region is known to have catastrophic consequences for corneal wound healing and integrity.

Keratocytes and the Cornea:

The cornea's transparency and refractive properties, necessary for clear vision, are maintained by the highly organized corneal stroma. Keratocytes are the cellular components of the stroma, and are also referred to as “corneal fibroblasts” and “stromal cells of the cornea”. As modified fibroblasts, keratocytes are responsible for the embryonic formation of the corneal stroma; and for postnatal maintenance of the stroma in a healthy eye. Keratocytes are also important in wound healing following corneal trauma.

Following corneal injury, damaged epithelial cells release interleukin-1 A (IL-1a) and interleukin-1 B (IL-1b), interleukins regulating a biochemical cascade involved in wound healing. See Wilson, SE et al., “Epithelial Injury Induces Keratocyte Apoptosis: Hypothesized Role for the Interleukin-1 System in the Modulation of Corneal Tissue Organization and Wound Healing,” Exp Eye Res; 1996. 62 (325-337). Although IL-1a and IL-1b aid in wound healing, the increase in concentrations of these interleukins in the stroma also results in a marked increase in keratocyte apoptosis (Id.). Apoptosis of keratocytes disrupts the highly organized structure of the stroma necessary for proper refraction of light by the cornea, and can result in anterior stromal haze. Changes in corneal keratocyte population density remain a common clinical problem in development; in aging; in corneal dystrophies including, eg., Fuchs' dystrophy, pseudophakik bullous keratopathy, and keratoconus; in changes in corneal clarity following excimer laser surgery, and following corneal grafting.

There is a need to develop a surgical graft comprising keratocytes for use in the treatment of corneal injury and corneal dystrophies. Further, to investigate how keratocytes maintain corneal stromal transparency, it is important to expand their number by sub-culturing. The inventors of the disclosed subject matter sought to develop a new method of expanding mesenchymal cells such as, for example, human corneal keratocytes, in serum while maintaining their characteristic phenotype.

Current Methods of Keratocyte Culture Using Serum:

Previously reported attempts failed to maintain the normal phenotype of keratocytes. For example, when cultured on a plastic substrate in a serum-containing medium, bovine and rabbit keratocytes rapidly lose their dendritic morphology, and acquire a fibroblastic morphology. See Beals M P, Funderburgh J L, Jester J V, Hassell J R, “Proteoglycan synthesis by bovine keratocytes and corneal fibroblasts: Maintenance of the keratocyte phenotype in culture,” Invest Ophthalmol Vis Sci 1999;40:1658-63. See also Jester J V, Barry-Lane P A, Cavanagh H D, Petroll W M, “Induction of a-smooth muscle actin expression and myofibroblast transformation in cultured corneal keratocytes,” Cornea 1996;15:505-16. At the same time, the keratocytes start expressing integrin α5β1 and α-smooth muscle actin, a marker for myofibroblasts, especially when seeded at a low density. See Masur S K, Cheung J K H, Antohi S, “Identification of integrins in cultured corneal fibroblasts and in isolated keratocytes,” Invest Ophthalmol Vis Sci 1993;34:2690-8. See also Desmouliére A, Geinoz A, Gabbiani F, Gabbiani G., “Transforming growth factor β-1 induces a-smooth muscle actin expression in granulation tissue myofibroblasts and in quiescent and growing cultured fibroblasts,” J Cell Biol 1993; 122:103-11; Gabbiani G. Chaponnier C, Hüttner I. Cytoplasmic, “Filaments and gap junctions in epithelial cells and myofibroblasts during wound healing,” J Cell Biol 1978;76:561-8; and Masur S K, Dewal H S, Dinh T T, et al, “Myofibroblasts differentiate from fibroblasts when plated at low density,” Proc Natl Acad Sci USA 1996; 93:4219-23. In addition, such culturing condition reduces the ratio of keratan sulfate-containing proteoglycans to dermatan sulfate-containing proteoglycans. See Beals, et. al., Supra; Dahl, et. al., “The synthesis of glycosaminoglycans by corneal stroma cells in culture,” Exp Cell Res 1974;88:193-7; and Dahl I M, Coster L., “Proteoglycan biosynthesis in cultures of corneas and corneal stroma cells from adult rabbits,” Exp Eye Res 1978;27:175-90.

Current Methods of Keratocyte Culture Without Serum:

Without knowing what factor or factors in the serum may be detrimental to the maintenance of the keratocyte phenotype, a serum-free medium has been adopted to culture bovine keratocytes so as to maintain the characteristic dendritic morphology and a normal ratio of keratan sulfate-containing proteoglycans to dermatan sulfate-containing proteoglycans. See Beals, et. al., Supra. Under such a serum-free culturing condition, these keratocytes secrete lumican, keratocan and mimecan. See Berryhill, et. al., “Production of prostaglandin D synthase as a keratan sulfate proteoglycan by cultured bovine keratocytes,” Invest Ophthalmol Vis Sci 2001 ;42:1201-7; and Berryhill, et. al., “Partial restoration of the keratocyte phenotype to bovine keratocytes made fibroblastic by serum,” Invest Ophthalmol Vis Sci 2002;43:3416-21. It is important to note, however, that this serum-free culturing method precludes ex vivo expansion and sub-culturing. See Beals, et. al., Supra.; and Jester et. Al., “Corneal stromal wound healing in refractive surgery: the role of myofibroblasts,” Prog Retin Eye Res 1999;18:3111-56.

Based on the foregoing, it is believed that prior to the present invention, the culturing of keratocytes wherein the normal phenotype, for example, the dendritic morphology of the keratocytes, and the function of the keratocytes, including their continuous expression of keratocan, even in the presence of high concentrations of serum, was unknown. Thus, without serum, keratocytes could not be expanded ex vivo; and with serum, the normal dendritic morphology and the function of the keratocytes could not be maintained.

Ongoing Need for Improved Surgical Grafts Comprising Limbal Tissue:

The transplantation of limbal tissue can replenish SC population to support regeneration of the entire corneal surface epithelium. See Kenyon K R, Tseng S C G, “Limbal autograft transplantation for ocular surface disorders,” Ophthalmology. 1989;96:709-723; and Tsai R J F, Sun T-T, Tseng S C G, “Comparison of limbal and conjunctival autograft transplantation for corneal surface reconstruction in rabbits,” Ophthalmology. 1990; 97:446-455. There is an ongoing need to develop an improved surgical graft for use in the transplantation of limbal tissue. There is also a need to develop a consistent and reproducible method of isolating an intact and viable limbal epithelial sheet including the basal epithelium. Prior to the present invention, there appears to have been no reported demonstration of the complete removal of an intact viable human limbal epithelial sheet.

### SUMMARY OF THE INVENTION

The invention inter alia includes the following, alone or in combination. In one aspect, the present invention relates to our discovery of a method of enzymatically, consistently and reproducibly, isolating a viable, intact limbal epithelial sheet that retains stem cell characteristics in the basal epithelium. According to an embodiment of the invention, a method of enzymatically isolating a substantially intact, viable limbal epithelial sheet comprises the steps of: a) obtaining limbus from a biopsy of a donor eye chosen from an eye of a living individual, an eye of a living, related individual, and a cadaveric eye, the limbus comprising limbal epithelium and an underlying stroma; b) contacting the limbus with a solution comprising Dispase 2, for a period of time and under conditions sufficient to loosen a limbal epithelial sheet from the stroma, thereby forming a loosely adherent limbal epithelial sheet; and c) mechanically separating the loose epithelial sheet from the underlying stroma, thereby isolating a substantially intact, viable, limbal epithelial sheet.

One embodiment of the invention is a surgical graft comprising an isolated, substantially intact, viable, limbal epithelial sheet, the limbal epithelial sheet prepared by a process comprising the steps of: a) obtaining limbus from a biopsy of a donor eye of an individual or a cadaveric eye, the limbus comprising limbal epithelium and an underlying stroma; b)contacting the limbus with a solution comprising Dispase 2 for a period of time and under conditions sufficient to loosen a limbal epithelial sheet from the stroma, thereby forming a loosely adherent limbal epithelial sheet; and c) mechanically separating the loose epithelial sheet from the underlying stroma, thereby forming a surgical graft comprising an isolated, substantially intact, viable, limbal epithelial sheet. Another embodiment of the disclosed invention is a surgical graft comprising an isolated, substantially intact, viable, limbal epithelial sheet.

The invention also relates to a method of expanding ex vivo epithelial stem cells present in a limbal epithelial sheet, comprising the steps of: a) enzymatically isolating a limbal epithelial sheet according to the method of claim 1; b) contacting the limbal epithelial sheet with a basement membrane side of an amniotic membrane, thereby forming a composite comprising limbal epithelial sheet and amniotic membrane; and c) culturing the composite for a period of time and under conditions sufficient to enable the epithelial stem cells to expand.

In one aspect, the present invention relates to our discovery of a new culture system to achieve ex vivo expansion of human corneal keratocytes while maintaining their characteristic dendritic morphology and continuous expression of keratocan even in the presence of high concentrations of serum by growing them on the stromal matrix of the human amniotic membrane (AM). One embodiment of the invention is a surgical graft comprising keratocytes on amniotic membrane.

A method of expanding mesenchymal cells ex vivo, while maintaining the phenotype of the mesenchymal cells, has now been found; the method comprising: a) contacting a stromal side of an amniotic membrane with at least one type of mesenchymal cells, thereby forming a composite comprising the mesenchymal cells and the amniotic membrane; and b) culturing the composite in a serum-containing medium for a period of time and under conditions sufficient to enable the mesenchymal cells to expand while maintaining the phenotype of the mesenchymal cells.

The present invention has many advantages. Limbal tissue, and limbal tissue separated according to a method of the invention can comprise a surgical graft; can be used to replenish a stem cell population; and can be used to support regeneration of the entire corneal surface epithelium. A limbal epithelial sheet generated according to the disclosed method can also be used in tissue engineering, specifically for ex vivo expansion of epithelial stem cells.

An enzymatic isolation of a limbal epithelial sheet according to an embodiment of the method of the invention is useful as a method by itself, to produce an intact, viable limbal sheet for the study of limbal stem cells; and as a novel way of separating a limbal epithelial sheet in order to remove stem cells from the limbus or to retain stem cell characteristics in the basal epithelium.-The sheet can also be used to facilitate the purification of limbal stem cells, once the surface marker has been identified.

A composite comprising amniotic membrane and keratocytes, according to an embodiment of the invention can be used as a surgical graft to replenish a keratocyte population, and to support regeneration of damaged corneal stroma. Even in the presence of high serum, the keratocytes cultured on amniotic membrane retain their phenotype and dendritic morphology. Other mesenchymal cells also maintain their phenotype when expanded in high serum on amniotic membrane.

A composite comprising amniotic membrane and keratocytes or other mesenchymal cells according to an embodiment of the invention can also be used in tissue engineering, specifically for ex vivo expansion of keratocytes or other mesenchymal cells.

### BRIEF DESCRIPTION OF THE DRAWINGS

The foregoing and other objects, features and advantages of the invention will be apparent from the following more particular description of illustrative embodiments of the invention, as illustrated in the accompanying drawings. The drawings are photographs, emphasis being placed upon illustrating the results of exemplary embodiments of the disclosed method.

FIGS. 1A and 1C are photographs illustrating the amount of pigmentation in two different corneoscleral rims used in this study.

FIGS. 1B, 1D, and 1E are micrographs showing the Palisades of Vogt are clearly distinguishable at higher magnifications.

FIGS. 2A and 2B are micrographs of examples of two entire limbal epithelial sheets isolated using the described method. The normal architecture of palisades of Vogt was maintained after removal.

FIG. 2C is a phase contrast microscopic view of large and flat superficial cells of the limbal epithelial sheet surface.

FIG. 2D is a phase contrast microscopic view of small rounded cells at the bottom of the epithelial sheet. (Scale bar=40 μm)

FIG. 3A is a micrograph of Hematoxylin staining showing an isolated limbal epithelial sheet with flat superficial cells, intermediate wing cells, and small occasionally pigmented cells in the basal layer. (Inset shows a longer segment of the limbal sheet at a low magnification (100×)).

FIG. 3B is a micrograph of another isolated limbal sheet showing an undulating inferior border as seen in vivo.

FIG. 3C is a micrograph showing full thickness staining by an anti-keratin 3 antibody in the peripheral cornea (right of the arrow), but supra-basal staining in the limbal epithelium (left of the arrow).

FIG. 3D is a higher magnification view showing the absence of anti-keratin 3 staining in the basal region of the limbal sheet.

FIG. 3E is a micrograph showing Connexin 43 staining was positive in intercellular junctions of suprabasal epithelial cells.

FIG. 3F is a micrograph showing that the basal positive p63 staining was observed in the entire limbal sheet. (Scale bar=40 μm)

FIG. 4A is a micrograph wherein Hematoxylin staining shows a loosely adherent limbal epithelial sheet as evidenced by the spaces created in between the epithelium and the underlying stroma, the spaces marked by asterisks.

FIG. 4B is a micrograph wherein Collagen IV shows positive in the blood vessels and the superficial stroma of the limbus with a discontinuous staining in the basal surface of the loose limbal epithelial sheet.

FIG. 4C is a micrograph wherein Collagen VII shows a positive lineal staining in the superficial stroma of the corneal portion, but is weak in that of the limbus after digestion.

FIG. 4D is a higher magnification view of the peripheral cornea showing a lineal staining for collagen VII.

FIG. 4E shows, however, collagen VII is diffuse in the superficial stroma of the limbus.

FIG. 4F shows negative laminin 5 staining in the limbus after digestion. (Scale bar=40 μm)

FIG. 5A is a micrograph showing a linearly positive staining to integrin β4 is present on the basal epithelial cell surface of the isolated sheets.

FIG. 5B shows negative laminin 5 staining was noted in the entire epithelial sheet.

FIG. 5C shows Collagen IV was sporadically positive on the basal surface of isolated sheets.

FIG. 5D shows that no collagen VII was observed in any isolated sheet.

FIG. 5E shows that hematoxylin staining of a remaining stroma indicates the absence of epithelial cells.

FIG. 5F shows Collagen IV is strongly positive in a lineal pattern on the superficial surface of the remaining limbal stroma.

FIG. 5G shows Collagen VII was diffusely positive in the superficial stroma of the stromal remnant. (Scale bar=40μm)

FIG. 6A is a photograph of cultures of two different limbal epithelial sheets (1.5 mm arc length) cultured until confluency and stained with crystal violet.

FIG. 6B is a side elevational view of the two cultures in 6A, the side view showing the extent of the outgrowth on the dish wall.

FIGS. 6C and 6D are views of phase contrast microscopy showing a monolayer of small compact epithelial cells.

FIG. 6E is a photograph of the results of Western blot analysis of the proteins extracted from expanded cells, the photograph showing a band at 60 kDa (p63) and another band at 64 kDa (keratin 3).

FIGS. 7A-D show morphological differences in Primary Plastic and AM Cultures.

After one week culturing, cells were dendritic and formed intercellular networks when cultured on AM stroma in 1% FBS (Fig. A) or 10% FBS (Fig.B)

FIG. 7C (Prior Art) micrograph shows, in contrast, cells cultured on plastic appeared stellate and spindle in 1% FBS.

FIG. 7D (Prior Art) micrograph shows cells appear uniformly spindle after rapid growth on plastic with 10% FBS.

All micrographs are taken at the same magnification. (Bar, 25 μm.)

FIG. 8(A-D) shows Cytoplasmic Staining Using LIVE AND DEATH ASSAY®.

FIG. 8A and FIG. 8B are micrographs showing that in primary AM cultures, cells formed extensive intercellular contacts (Fig.A) with some showing extensive dendritic processes projected in three dimensions (Fig.B).

FIG. 8C and FIG. 8D (both Prior Art) show, in contrast, that cells in primary plastic cultures did not form intercellular contacts (Fig.C), and appeared spindle without dendritic processes (Fig.D). Micrographs A and C were taken at the same magnification, while micrographs B and D were taken at the higher magnification. (Bar, 25 μm.)

FIG. 9A-9F shows morphological difference between Plastic and AM Cultures after sub-culturing. Cells cultured on AM continued to show dendritic morphology at passage 2 (FIG. 9A) and passage 4 (FIG. 9C) and maintain extensive intercellular contacts (FIG. 9B and FIG. 9D, respectively). In contrast, cells cultured on AM at passage 1 immediately adopted a fibroblastic morphology when they were subcultured on plastic, (FIG. 9E-Prior Art) with no intercellular contacts (9F-Prior Art). All micrographs are taken at the same magnification. (Bar, 25 μm.)

FIG. 10A-10C). Changes in Morphology and Keratocan Expression. Cells that have continuously been subcultured on plastic for 3 passages were seeded on plastic (FIG. 10 A Prior art) and AM (FIG. 10B). The fibroblastic morphology noted on plastic (FIG. 10A-Prior Art) remained the same and did not revert to a dendritic morphology when seeded on AM (FIG. 10-B). Micrographs are taken at the same magnification. (Bar, 25 μm.) FIG. 10C shows expression of keratocan transcript.

FIG. 11. RT-PCR Analysis of Expression of Keratocan Transcript in Primary Cultures. Total RNA was extracted from primary plastic and AM cultures. Using GAPDH (573 bp) as a loading control, expression of keratocan transcript (1059 base pair (bp)) was barely detected on plastic with 1% FBS, but absent in 5% or 10% FBS. In contrast, keratocan transcript was readily detected in cells on AM in 1%, 5% and 10% FBS (with the highest noted in 5% FBS) and in normal corneal stroma (K).

FIG. 12 shows RT-PCR Analysis of Keratocan, Lumican, and Collagen 111-a1 Transcripts. Total RNA was extracted from AM and plastic cultures (Prior Art) for up to passage 5.

FIG. 13 is a photograph showing the results of Western Blotting Analysis of Keratocan Protein. A 50 KD protein band corresponding to de-glycosylated keratocan was detected in the normal corneal stroma (K) and cells grown on AM, but not detected in cells grown on (Prior Art) plastic at passages 2 and 4.

### DETAILED DESCRIPTION OF THE INVENTION

A description of preferred embodiments of the invention follows. It will be understood that the particular embodiments of the invention are shown by way of illustration and not as limitations of the invention. At the outset, the invention is described in its broadest overall aspects, with a more detailed description following. The features and other details of the compositions and methods of the invention will be further pointed out in the claims.

Disclosed herein are surgical grafts, including autografts and allografts, and methods of preparation thereof. An autograft is a graft prepared from the recipient's own tissue, for example from a healthy eye of the recipient. An allograft is a graft of tissue between individuals who are not genetically identical. An allograft may be prepared from tissue obtained from a cadaveric eye or living-related individual, for example. A disclosed surgical graft may comprise amniotic membrane. In one embodiment, the amniotic membrane has cells and an extracellular matrix; and wherein prior to using the amniotic membrane as a surgical graft the cells of the amniotic membrane have been killed while maintaining the integrity of the extracellular matrix. Amniotic membrane suitable for use in an embodiment of the present invention may be prepared as described, for example in U.S. Pat. No. 6,152,142 to Tseng, and in U.S. Pat. No. 6,326,019 B1 to Tseng, the teachings of both of which are incorporated herein by reference in their entirety.

In one embodiment the surgical graft comprises amniotic membrane that has been freeze-dried prior to using the amniotic membrane as a surgical graft. Techniques suitable for freeze-drying amniotic membrane are well known to those of skill in the art of tissue preparation.

A method of expanding mesenchymal cells such as keratocytes, ex vivo, while maintaining the phenotype of the mesenchymal cells, has now been discovered. The disclosed method includes the use of amniotic membrane as a substrate for the culture of keratocytes and other mesenchymal cells.

Prior to the present invention, the isolation and removal of an intact, viable sheet of limbal epithelial cells appears to be unknown. Herein is disclosed our new technique of isolating a substantially intact and viable human limbal epithelial sheet using Dispase 2 under a special digestion protocol. Dispase 2, also referred to as Dispase II, is a neutral protease produced by Bacillus polymyxa. We have further characterized the cleavage plane and reported unique findings different from those of ethanol treatment used in laser-assisted epithelial keratomieulesis (LASEK). The significance of this new isolation technique is further discussed. The present invention provides, inter alia a method of enzymatically isolating an intact, viable limbal epithelial sheet that retains certain stem cell characteristics. The limbal epithelial sheet according to an embodiment is substantially intact and viable. “Substantially intact”, as the term is used herein, means that the epithelial cells are connected or integrated to form a sheet-like layer, and appear microscopically as a sheet of cells, even though a few cells may be disconnected from the sheet at any given time. “Substantially intact” and “intact”, as the terms are used herein, have the same meaning and are used interchangeably.

The limbal epithelial sheet isolated according to a method of the invention loosely adheres to the underlying stroma. The terms “loose,” “loosely adheres,” “loosely adherent,” and grammatical variations thereof, mean that the cells, epithelial sheet, or other tissue so described is more easily removed from the underlying stroma or other tissue than are cells, epithelial sheet, or other tissue not described as “loose” or “loosely adherent.” Contact with Dispase 2 for a sufficient period of time loosens the sheet so that it can be easily removed by mechanical means. The expression “to loosen a limbal epithelial sheet from the stroma” means to sufficiently overcome some of the attractive or bonding forces that cause the epithelial sheet to adhere to the stroma. The removal of a limbal epithelial sheet according to a method of the invention results in a cleavage plane that is within the basement membrane zone, and as a result, the cell membrane and intracellular junctions remain intact, protecting the cells from damage and possibly contributing to the maintenance of normal cellular phenotype, as described below.

The method of enzymatically isolating a substantially intact, viable limbal epithelial sheet disclosed herein comprises the steps of: a) obtaining limbus from a biopsy of a donor eye of a living individual, an eye of a living, related individual, or from a cadaveric eye, the limbus comprising limbal epithelium and an underlying stroma; b)contacting the limbus with a solution comprising Dispase 2, for a period of time and under conditions sufficient to loosen a limbal epithelial sheet from the stroma, thereby forming a loosely adherent limbal epithelial sheet; and c) mechanically separating the loose epithelial sheet from the underlying stroma, thereby isolating a substantially intact, viable, limbal epithelial sheet.

In one embodiment of the disclosed method, the solution contacting the limbus according to step b) further comprises a substance chosen from SHEM, a polyhydroxy alcohol, a sugar, and combinations thereof. Examples of a polyhydroxy alcohol suitable for use in an embodiment include sorbitol, mannitol, and galactitol. In an exemplary embodiment, the conditions of step b) include maintaining a temperature from about 0° C. to about 37° C. for at least half an hour. Other exemplary embodiments are disclosed in the examples below.

One embodiment of the invention is a surgical graft, that may be an allograft or an autograft, comprising an isolated, substantially intact, viable, limbal epithelial sheet. The limbal epithelial sheet may retain at least one stem cell characteristic. In one embodiment the limbal epithelial sheet comprises cells that do not express keratin 3, or connexin 43, but may express p63.

The surgical graft comprising a limbal epithelial sheet may further comprise amniotic membrane. In an exemplary embodiment the amniotic membrane has cells and an extracellular matrix; and wherein prior to using the amniotic membrane as a surgical graft the cells of the amniotic membrane have been killed while maintaining the integrity of the extracellular matrix. In another embodiment, the disclosed surgical graft comprising limbal epithelial sheet further comprises amniotic membrane that has been freeze-dried prior to using it as a surgical graft, and limbal epithelial sheet.

Disclosed in the Summary above and in the detailed examples below is a method of expanding ex vivo epithelial stem cells present in a limbal epithelial sheet, comprising the steps of: a) enzymatically isolating a limbal epithelial sheet according to the method disclosed herein; b) contacting the limbal epithelial sheet with a basement membrane side of an amniotic membrane, thereby forming a composite comprising limbal epithelial sheet and amniotic membrane; and c) culturing the composite for a period of time and under conditions sufficient to enable the epithelial stem cells to expand. As used herein, the term “expand” refers to the growth of cells in culture, the growth resulting in an increase in the number of cells in the culture.

One embodiment of the invention is a surgical graft comprising limbal epithelial cells and limbal epithelial stem cells expanded ex vivo according to the method disclosed herein. In one embodiment, the surgical graft comprises amniotic membrane having cells and an extracellular matrix; and wherein prior to using the amniotic membrane as a surgical graft the cells of the amniotic membrane have been killed while maintaining the integrity of the extracellular matrix. In another embodiment, the surgical graft comprises amniotic membrane that has been freeze-dried prior to using the amniotic membrane as a surgical graft. In one embodiment the graft is an allograft. In another embodiment the graft is an autograft.

In yet another embodiment of the invention the surgical graft may further comprise mesenchymal cells. Examples of mesenchymal cells suitable for use in a graft incliude keratocytes. Other mesenchymal cells can be chosen from fetal mesenchymal cells, keratocytes, fibroblasts, endothelial cells, melanocytes, cartilage cells, bone cells, hematopoietic stem cells, bone marrow mesenchymal stem cells, adult mesenchymal stem cells, and combinations thereof.

Disclosed in the Summary above and in the detailed examples below is a method of expanding mesenchymal cells ex vivo, while maintaining the phenotype of the mesenchymal cells; the method comprising: a) contacting a stromal side of an amniotic membrane with at least one type of mesenchymal cells, thereby forming a composite comprising the mesenchymal cells and the amniotic membrane; and b) culturing the composite in a serum-containing medium for a period of time and under conditions sufficient to enable the mesenchymal cells to expand while maintaining the phenotype of the mesenchymal cells.

A surgical graft comprising mesenchymal cells expanded ex vivo according to the above-described method is also disclosed. Examples of mesenchymal cells suitable for expansion are keratocytes. The keratocytes expanded on amniotic membrane according to an embodiment maintain their phenotype. For example, the keratocytes expanded on amniotic membrane maintain dendritic morphology and maintain keratocan expression. Other mesenchymal cells suitable for expansion on amniotic membrane and subsequent use on a surgical graft can be chosen from fetal mesenchymal cells, fibroblasts, endothelial cells, melanocytes, cartilage cells, bone cells, hematopoietic stem cells, bone marrow mesenchymal stem cells, adult mesenchymal stem cells, and combinations thereof.

Limbal Epithelial Sheet Isolation

Our technique of isolating the entire limbal epithelial sheet is based on digestion by Dispase 2, a neutral protease from Bacillus Polymyxa. In the skin, the proteolytic action of Dispase 2 is thought to target at fibronectin and collagen IV of the basement membrane. Spurr and Gipson demonstrated disappearance of immunoreactivity to laminin in the rabbit cornea after 6 hours of incubation with 2.4 U Dispase 2 at 37° C. (See Spurr S J, Gipson I K, “Isolation of corneal epithelium with Dispase II or EDTA. Effects on the basement membrane zone,” Invest Ophthalmol Vis Sci. 1985;26:818-827.)

We noted that 18 h incubation of 50 mg/ml Dispase 2 at 4° C. degraded completely laminin 5 and the majority of collagen IV of the corneal and limbal basement membranes. In addition, such digestion regimen did not degrade collagen VII that forms anchoring fibrils in the corneal basement membrane, but caused their complete dissembly in the limbal basement membrane. Dispase 2 did not alter integrin β4 of the basal epithelium. Taken together, these findings support that the cleavage plane created by Dispase 2 is at the lamina densa of the basement membrane. The differences in the composition and anatomy of the limbal and central corneal basement membranes may explain why a different digestion regimen is needed to separate an intact human limbal epithelium from the stroma. (See Gipson I K, “The epithelial basement membrane zone of the limbus,” Eye, 1989;3 (Pt 2):132-140. See also Ljubimov, et. al., “Human corneal basement membrane heterogeneitiy: topographical differences in the expression of type IV collagen and laminin isoforms,” Lab Invest. 1995;72:461-473.)

The digestion by Dispase 2 will have to be extended to 18 hours in order to remove completely the limbal epithelial sheet. This notion was verified by the lack of epithelial outgrowth from the remaining stroma after sub-cultured for 2 weeks. In contrast, we noted that some limbal basal epithelial cells remained in the stroma when the same Dispase 2 dose was incubated at 37° C. for 1 hour or at 4° C. for 14 hours (not shown). Because it was necessary to incubate in Dispase 2 for such a long period of time, it is important to keep it at a low temperature (4° C.) to reduce metabolic activity, and maintain the tissue in a medium with growth supplements to maintain the viability and in the presence of 100 mM sorbitol to prevent cell swelling by increasing the osmolarity. (See Pfefer B A, “Improved methodology for cell culture of human and monkey retinal pigment epithelium,” Prog Retin Eye Res. 1.991;10:251-291, the entire teachings of which are incorporated herein by reference.) By doing so, we confirmed that isolated limbal epithelial sheets indeed retained a high viability of 80.7%. Because human limbal rings used in this study were not fresh and were studied after variable times following death, storing in an Eyebank Storage Medium, and transport to the laboratory, there might have been cell death prior to our digestion. Therefore, we have applied the same digestion protocol to a number of fresh pigmented rabbit limbus, and obtained a mean high viability of 93%.

Because the cleavage plane is within the basement membrane zone, the cell membrane and intercellular junctions remained intact. This not only protected cells from damage, but also maintained the normal cellular phenotype by preserving such intercellular structures as cadherins, integrins, and connexins. In this study, we noted that the basal epithelium of the isolated limbal epithelium retained pigmentation, did not express keratin 3 and connexin 43, but actively expressed p63. These characteristics are identical to SC features reported in human in vivo limbal epithelium.(See Schermer et. Al, “Differentiation-related expression of a major 64K corneal keratin in vivo and in culture suggests limbal location of corneal epithelial stem cells,” J Cell Biol. 1986;103:49-62. See also Pellegrini, et. al., “p63 identifies keratinocyte stem cells,” Proc Natl Acad Sci USA. 2001;98:3156-3161; and Matic, et. al., “Stem cells of the corneal epithelium lack connexins and metabolite transfer capacity,” Differentiation, 1997;61:251-260. )

The cleavage plane created by this dispase digestion is different from a brief treatment of 20% ethanol used to prepare an epithelial flap before excimer laser ablation in the procedure of LASEK, of which the cleavage plane is characterized to be located in the lamina lucida and the hemidesmosomes of the basement membrane, and there is ethanol induced cell membrane damage. (See Espana, et. al., “Cleavage of corneal basement membrane components by ethanol exposure like LASEK,” J Cat Refract Surg. 2003; in press.) To further verify that isolated limbal epithelial sheets indeed were viable and functioned properly, we seeded a small fragment of 1.5 mm arc length at the center of a 60 mm plastic dish, which rapidly grew into a confluent monolayer with small and compact epithelial cells. Furthermore, the final epithelial outgrowth expressed keratin 3 and p63, of which both are regarded as differentiation and proliferation markers of the limbal epithelial SC, respectively. (See Pellegrini G, et. al., “p63 identifies keratinocyte stem cells,” Proc Natl Acad Sci USA. 2001;98:3156-3161.)

With such an intact and viable epithelial sheet isolated from the human limbus, one may begin to study limbal SC with respect to their properties, proliferation and differentiation into the corneal epithelium, and their interaction with the underlying stromal niche. This technique may also facilitate the purification of limbal SC once the surface marker has been identified. Furthermore, it might also be useful to use isolated limbal epithelial sheet to expand limbal epithelial SC ex vivo for therapeutic epithelial transplantation.

Human Keratocytes Expanded on Amniotic Membrane.

The extracellular matrix of the corneal stroma contains a dense network of collagen fibrils and proteoglycans arranged in an order to allow transparency for clear vision. Keratocytes, i.e., cells in the corneal stromal matrix, are dendritic in shape, form extensive intercellular contacts; and synthesize the collagens I, V, VI, and XII, and keratan sulfate-containing proteoglycans such as lumican, keratocan, and mimecan. See Birk D E, et. al., “Collagen and glycosaminoglycan synthesis in aging human keratocyte cultures,” Exp Eye Res, 1981;32:331-9. See also Cintron C, Hong B S, “Heterogeneity of collagens in rabbit cornea: type VI collagen,” Invest Ophthalmol Vis Sci,988;29:760-6; and Funderburgh J L, Conrad G W, “Isoforms of corneal keratan sulfate proteoglycan,” J Biol Chem, 1990;265:8297-303.

Keratocan is the only keratan sulfate-containing proteoglycan synthesized by mouse keratocytes in vivo while lumican and mimecan are widely distributed. See Liu C-Y, et al., “The cloning of mouse keratocan cDNA and genomic DNA and the characterization of its expression during eye development,” J Biol Chem 1999;273 :22584-8.

We have now discovered a new culture system to achieve ex vivo expansion of human corneal keratocytes and other mesenchymal cells while maintaining their characteristic phenotype even in the presence of high concentrations of serum by growing them on the stromal matrix of the human amniotic membrane (AM). The phenotype maintained by the keratocytes expanded according to an embodiment of the disclosed method may include at least one of dendritic morphology and keratocan expression.

Herein we provide strong experimental evidence proving that dendritic morphology and keratocan expression by cultured human keratocytes can be maintained on AM stromal matrix during their continuous expansion in the presence of high concentrations of serum for at least 6 passages. This accomplishment represents a significant advance in the field of keratocyte biology because all previous attempts based on conventional plastic cultures have failed to do so. Earlier studies used the dendritic morphology and extensive intercellular contacts as the hallmark of keratocytes. See Beals M P, et al., “Proteoglycan synthesis by bovine keratocytes and corneal fibroblasts: Maintenance of the keratocyte phenotype in culture,” Invest Ophthalmol Vis Sci 1999;40:1658-63. Such a characteristic dendritic morphology can be achieved on plastic culture only in a serum-free medium, but is rapidly lost in a serum-containing medium. See Id., and Jester J V, et al, “Induction of a-smooth muscle actin expression and myofibroblast transformation in cultured corneal keratocytes,” Cornea 1996;15:505-16. Because AM stromal matrix continues to maintain such a characteristic morphology even in the presence of serum, this new culture system can allow ex vivo expansion of keratocytes for further manipulations and studies without losing its phenotype. As a result, we believe that this new culture system can be used as the first step toward engineering the human corneal stroma.

Our study also demonstrated that the dendritic morphology correlated well with the expression of keratocan transcript and protein. Among all keratan sulfate-containing proteoglycans, keratocan is uniquely expressed by keratocytes. See Pellegata N S, et al., “Mutations in KERA, encoding keratocan, cause cornea plana,” Nat Genet, 2000;25:91-5. Unlike lumican and collagen III-a1, which were uniformly expressed by cells on both plastic and AM, keratocan was expressed only by cells on AM. This finding further supports the notion that keratocan expression is a specific hallmark for keratocytes. This new culture system based on AM stromal matrix will help us investigate how keratocan gene is expressed and determine whether expression of keratocan influences the corneal stromal transparency.

It is worth being reiterated that the phenotype of keratocytes with respect to dendritic morphology and keratocan expression is easily lost on plastic when serum is added, but can be maintained on AM even in the presence of high serum. Such a contrast in serum modulation provides a clue from which one might probe the mechanism by which keratocyte phenotype is maintained.

Exemplary embodiments of the methods and composites of the present invention are described in detail in the examples below, and may be modified using readily available starting materials, reagents, and conventional laboratory procedures.

### EXAMPLE 1

(A Reproducible Method of Isolating an Intact Viable Human Limbal Epithelial Sheet; Culturing; and Characterization of Results.)

Materials and Methods:

Human pigmented limbus was incubated at 4° C. for 18 h in SHEM containing 50 mg/ml Dispase 2 and 100 mM sorbitol. A loose limbal epithelial sheet was separated by a spatula. The remaining stroma was digested and subcultured. Viability of isolated cells was assessed. Isolated epithelial sheets and remaining stroma were subjected to immunostaining. Sheets of 1.5 mm length were cultured in SHEM on plastic until confluency and cell extracts were subjected to Western blotting.

Results:

Intact limbal epithelial sheets were consistently isolated. Pigmented palisades of Vogt revealed large superficial squamous cells and small basal cuboidal cells. No epithelial cells grew from the remaining stroma. Mean viability was 80.7±9.1%. The basal epithelium was negative to keratin 3 and connexin 43, but was scatter positive to p63. The epithelial sheet showed negative staining to laminin 5 and collagen VII, but interrupted linear basal staining to collagen IV. The remaining stroma showed negative staining to laminin 5, positive linear staining to collagen IV in the basement membrane, and diffuse staining to collagen VII in the superior stroma subjacent to the basement membrane. Western blotting revealed that cells originated from the limbal sheets expressed keratin 3 and p63.

Conclusion:

An intact limbal epithelial sheet can be consistently and reproducibly isolated and contains stem cell characteristics in the basal epithelium by degrading laminin 5 and part of collagen IV, and disassembling collagen VII.

### EXAMPLE 2

(Enzymatic Isolation of Limbal Epithelial Sheets; Culture; Evaluation)

Materials:

Plastic cell culture dishes (60 mm) were from Falcon (Franklin Lakes, N.J., USA). Amphotericin B, Dulbecco's modified Eagle's medium (DMEM), F-12 nutrient mixture, fetal bovine serum (FBS), gentamicin, Hank's balanced salt solutions (HBSS), HEPES-buffer, neomycin, penicillin, streptomycin, phosphate buffered saline (PBS), TRIZOL® and 0.05% trypsin/0.53mM EDTA were purchased from Gibco-BRL (Grand Island, N.Y., USA). A LIVE/DEAD® viability/cytotoxity kit was from Molecular Probes (Eugene, Oreg., USA). Dispase 2 powder was obtained from Roche (Indianapolis, Ind., USA). Tissue-Tek OCT compound and cryomolds were from Sakura Finetek (Torrance, Calif., USA). Other reagents and chemicals including bovine serum albumin (BSA), cholera-toxin (subunit A), collagenase A, dimethyl sulfoxide, hydrocortisone, insulin-transferrin-sodium selenite (ITS) media supplement, mouse-derived epidermal growth factor (EGF), pre-stained broad band SDS-PAGE standard and sorbitol were purchased from Sigma (St. Louis, Mo., USA). An immunoperoxidase staining kit (Vecstating) and DAPI containing mounting media (Vectashield®) were obtained from Vector Laboratories (Burlingame, Calif., USA). We obtained the following monoclonal antibodies: keratin 3 (AE5) (ICN, Aurora, Ohio, USA), integrin □4 (Chemicon, Temeluca, Calif., USA), laminin 5 (Accurate Chemicals, Westbury, N.Y., USA), mouse anti collagen VII antibody, rhodamine conjugated rabbit anti-goat antibody and fluorescein-conjugated goat anti-mouse antibody (Sigma, St. Louis, Mo., USA) and a goat polyclonal antibody against collagen IV (Southern Biotech, Birmingham, Ala., USA).

Methods:

Enzymatic Isolation of Limbal Epithelial Sheets.

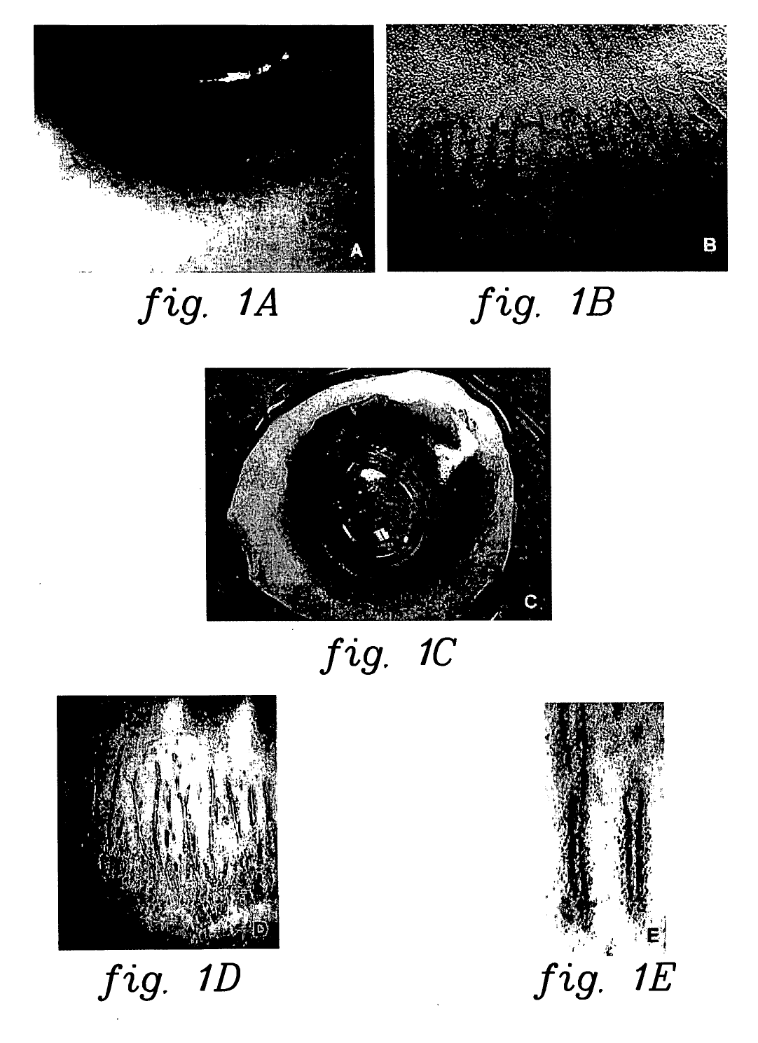

Twelve pigmented human corneoscleral rims from donors, younger than 50 years old and less than four days post harvesting, were obtained from the Florida Lions Eye bank within 8 hours after penetrating keratoplasty. FIGS. 1A and C show the amount of pigmentation in the selected rims and Figures B, D and E show a view of Vogt's palisades with a high magnification. After corneal transplantation, they were immediately transferred to SHEM medium, which was made of an equal volume of HEPES-buffered DMEM and Ham's F12 containing bicarbonate, 0.5% dimethyl sulfoxide, 2 ng/ml mouse derived EGF, 5 μg/ml insulin, 5 μg/ml transferrin, 5 ng/ml sodium selenite, 0.5 μg/ml hydrocortisone, 30 ng/ml cholera toxin A subunit, 5% FBS, 50 μg/ml gentamicin, and 1.25 μg/ml amphotericin B. They were then transported at 4° C. within 2 h to the laboratory, where the rims were rubbed off the endothelium and the uveal tissue using a cotton tip and cut by a razor blade into four symmetrical segments, each spanning 3 clock hours starting from 12 O'clock. Each segment was incubated at 4° C. in SHEM containing 50 mg/ ml Dispase 2 and 100 mM sorbitol for 18 h. Under a dissecting microscope, an already loose limbal epithelial sheet was separated by inserting and sliding a non-cutting flat stainless steel spatula into a plane between the limbal epithelium and the stroma. This maneuver was videophotographed.

Cell Culture of Remaining Stroma.

To determine if there was any epithelial cell left, a total of 16 remaining stromal segments from 8 different donor rims were incubated at 37° C. for 20 min in DMEM containing 1 mg/ml collagenase A. After centrifuge to remove the digestion solution, the remnants were cultured for 2 weeks at 37° C. in a DMEM medium containing 10% FBS, 20 mM HEPES, 50 μg/ml gentamicin and 1.25 μg/ml amphotericin B under 5% carbon dioxide humidified environment. The medium was changed every 2 to 3 days.

Five segments that were not exposed to Dispase 2 digestion were subjected to the same collagenase A digestion as described above and used as a positive control.

Viability Evaluation.

To determine the viability, 6 isolated limbal epithelial sheets from 6 different donor rims were incubated at 37° C. for 5 min in HBSS containing 0.05% trypsin and 0.53 mM EDTA. After a brief pipetting to achieve a single cell suspension, cells were centrifuged at 800×g for 5 min and re-suspended in PBS containing 2 μM calcein AM and 4 μM ethidium homodimer for 45 min at room temperature before cell counting under a fluorescent microscope. A mean percentage of live cells was calculated by counting both dead (red fluorescence) and live (green fluorescence) cells at ten different locations of a plastic dish. Cultured human corneal epithelial cells expanded from limbal explants that were exposed to methanol for 1 h were used as a positive control as dead cells. (See Tseng S C G, Zhang S-H, “Limbal epithelium is more resistant to 5-fluorouracil toxicity than corneal epithelium,” Cornea 1995; 14:394-401, the teachings of which are incorporated herein by reference in their entirety.)

Immunofluorescent Staining.

After incubating the corneoscleral rims in Dispase 2 as described above, one piece of corneoscleral rim without removing the epithelium was embedded in OCT and snap-frozen in liquid nitrogen for 5 μm frozen sectioning. As a comparison, epithelial sheets and remaining stroma were separately subjected to frozen sectioning. After fixation in cold acetone for 10 min at −20° C., immunofluorecence staining was performed as previously described, (See Grueterich M, Espana E, Tseng S C, “Connexin 43 expression and proliferation of human limbal epithelium on intact and denuded amniotic membrane,” Invest Ophthalmol Vis Sci. 2002;43:63-71, the teachings of which are incorporated herein by reference in their entirety.) using antibodies against the following antigens: keratin3 (1:100), connexin 43 (1:100), p63 (1:40), integrin β4 (1:100), collagen IV (1:50), collagen VII (1:100) and laminin 5 (1:100). The primary antibody was detected using a fluorescein conjugated secondary antibody except for collagen IV in which a rhodamine conjugated antibody was used. Sections were mounted in anti-fading solution containing DAPI VECTASHIELD® (Vector Laboratories, Burlingame, Calif., USA), and analyzed with a NikonTe-2000u Eclipse epi-fluorescent microscope (Nikon, Tokyo, Japan).

Characterization of Isolated Epithelial Sheet Outgrowth on Plastic.

Segments of isolated limbal epithelial sheets (n=7) of 1.5 mm of arc length were cultured until confluency in 60 mm dishes containing SHEM. To determine the expression of keratin 3, that is regarded as a corneal differentiation marker and p63 nuclear protein, that is a presumed corneal SC marker, proteins of confluent cultures were extracted by TRIZOL®, and precipitated by centrifuging at 12000×g in 100% isopropyl alcohol. After washing and centrifuge for three times, the protein pellet was precipitated with a solution of 95% ethanol containing 0.3 M guanidine hydrochloride. A final wash was performed with 100% ethanol and the protein pellet was air dried for 10 min. Pre-stained broad band SDS-PAGE standard and protein samples were dissolved into 1×SDS loading buffer: 50 mM Tris Cl, pH 6.8, 100 mM dithiothreitol, 2% SDS, 1% bromophenol blue and 10% glycerol. Ten pg of total proteins were electrophoresed in a 7.5% gradient polyacrylamide gel. After electrophoretic transfer to a nitrocellulose membrane, the membrane was immersed for 30 min in TTBS, which contained 0.1% (v/v) TWEEN 20™ in 100 mM Tris, 0.9% NaCl, pH 7.5, followed by 1 h blocking with 5% low fat dry milk in TTBS. TWEEN 20™, also known generically as Polysorbate 20, is a surfactant and spreading agent. Membranes were incubated for 1 h at room temperature with primary antibody against p63 (1:250 dilution) and keratin 3 (1:1000 dilution). After washing with TTBS, each membrane was transferred to a 1:200 diluted solution of biotinylated goat anti-mouse antibody in TTBS containing 1% horse serum. After incubating for 30 min, the membrane was incubated with 1:50 diluted VECTASTAIN ELITE® ABC reagent conjugated with peroxidase for 30 min and developed in diaminobenzidine (DAB) (DAKO, Carpintera, Calif., USA) between one and three min.

Results:

Isolation of Epithelial Sheet.

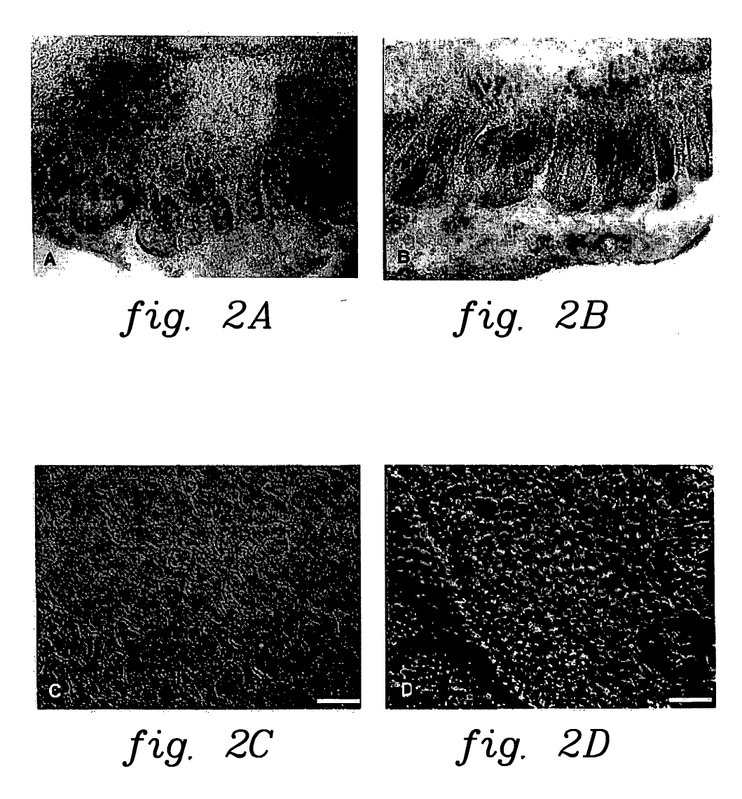

Intact limbal epithelial sheets were consistently removed from 48 limbal segments, demonstrating the procedure's simplicity and reproducibility. As shown in FIG. 2, the entire isolated limbal sheet with pigmented palisades of Vogt can be obtained (FIGS. 2A and 2B for example). Microscopic evaluation of the remaining limbal stromal surface revealed the lack of pigmented tissue (not shown). Phase contrast microscopic view of the isolated limbal sheets showed large superficial cells on the surface (FIG. 2C) and small basal epithelial cells on the basal surface of the sheet (FIG. 2D). The isolated limbal epithelial sheet was easy to handle and could be transferred to a culture dish in a medium using a transfer pipette to maintain the sheet's integrity in all cases.

Culturing the Stromal Remnants after Epithelial Sheet Removal.

No epithelial outgrowth was seen in any of 16 limbal stromal remnants that were digested by collagenase A and cultured for two weeks. Instead, abundant fibroblasts grew out of these stromal remnants in every remnant. In contrast, all 5 control samples with an intact limbal epithelium showed a characteristic epithelial outgrowth. These findings confirmed that there was no epithelial cell remaining on the stroma after the above isolation.

Cell Viability.

The isolated epithelial sheet was then subjected to a brief trypsin/EDTA treatment to render into single cell suspensions. The mean viability rate of six different samples was 80.7±9.1% (ranging from 66.3 to 90.7%). The positive control of methanol-treated cultured human corneal epithelial cells showed a viability of 0% (i.e., 100% of dead cells).

Characterization of Epithelial Phenotype of Isolated Limbal Sheets.

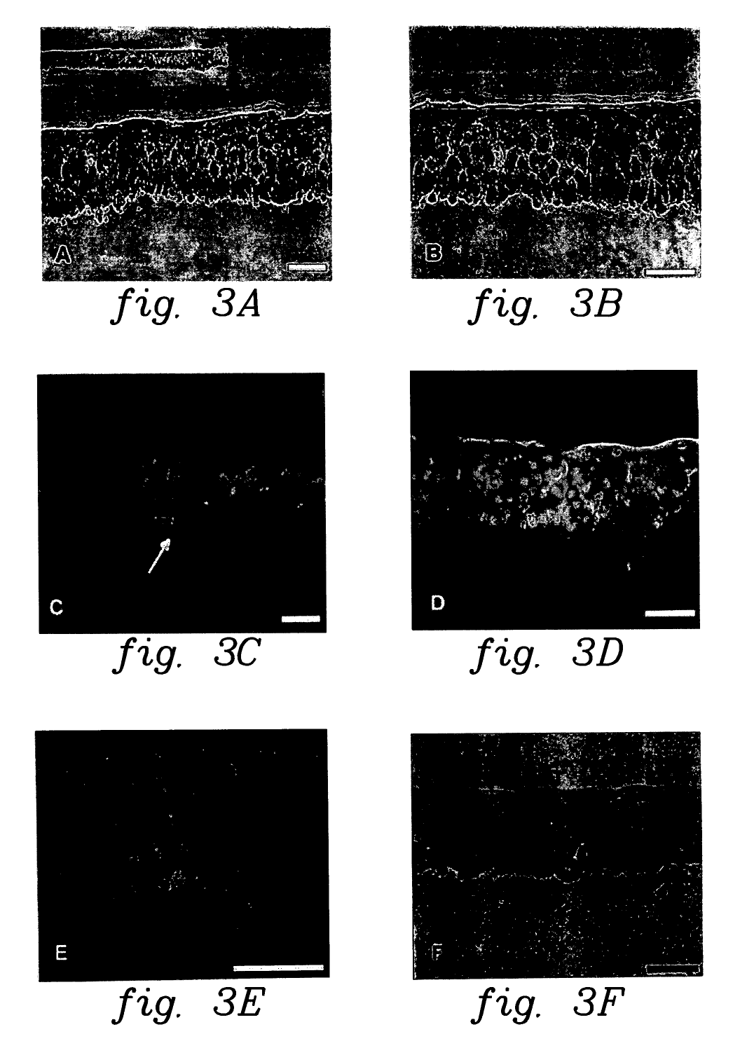

Hematoxylin staining of the isolated limbal epithelial sheet showed a stratified and organized epithelium identical to what has been noted in vivo human limbus. This stratified epithelium consisted of superficial large squamous cells, intermediate wing cells, and small basal epithelium, which was associated with pigmentation (FIG. 3A, showing the lower magnification of the entire sheet, and FIG. 3B). The superficial surface was smooth, while the basal surface was undulating. Immunostaining of the isolated limbal epithelial sheet showed strong intracytoplasmic staining to the AE-5 antibody, which recognizes keratin 3, in the full thickness stratified epithelium corresponding to the peripheral corneal epithelium (right of the arrow, FIG. 3C), and suprabasal cell layers of the limbal epithelium (left of the arrow, FIG. 3C, and FIG. 3D). This AE-5 staining pattern showing the basal negativity of keratin 3 has been reported as a proof of limbal epithelial SC. (See Schermer A, Galvin S, Sun T-T, “Differentiation-related expression of a major 64K corneal keratin in vivo and in culture suggests limbal location of corneal epithelial stem cells,” J Cell Biol. 1986; 103:49-62.) The intercellular punctuate staining of connexin 43 was found in the suprabasal cells, but absent in the basal cells (FIG. 3E), a pattern noted previously as well. (See Matic M, et al., “Stem cells of the corneal epithelium lack connexins and metabolite transfer capacity.” Differentiation 1997;61:251-260. See also Wolosin J M, et.al., “Stem cells and differentiation stages in the limbo-corneal epithelium,” Prog Retinal & Eye Res, 2000; 19:223-255.) The staining to the transcription factor p63, a reportedly limbal SC marker, was located exclusively in the basal cell layer (FIG. 3F). (See Pellegrini G, et. al., “p63 identifies keratinocyte stem cells,” Proc Natl Acad Sci USA 2001;98:3156-3161.)

Characterization of the Basement Membrane-Adhesion Complex After Complete Digestion.

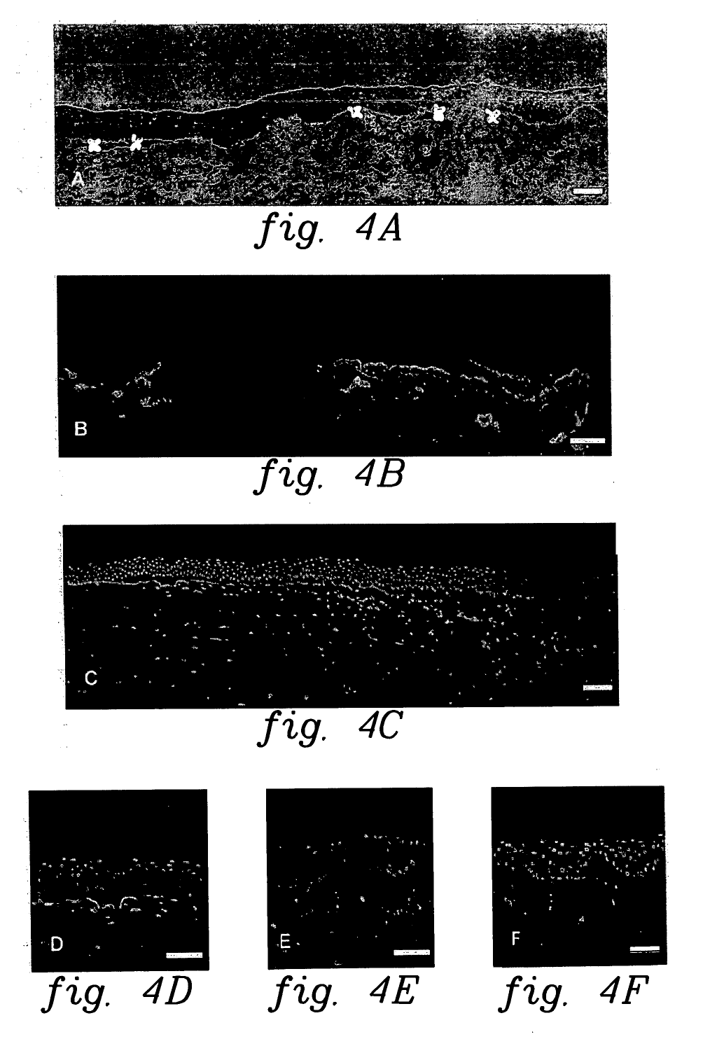

After complete dispase digestion, we analyzed the basement membrane-adhesion complex before the limbal epithelium was separated. Hematoxylin staining showed that the limbal epithelium was loosely adherent to the underlying stroma as evidenced by the spaces created in between (marked by * FIG. 4A). The staining to collagen IV was positive in the blood vessels and the superficial stroma of the limbus with discontinuous staining in the basement membrane area of the basal surface of the loose limbal epithelial sheet (FIG. 4B). The staining to collagen VII was linearly positive in the superficial stroma of the corneal portion, but was weak in the superficial stroma of the limbus after digestion (FIG. 4C). Under a higher magnification, the strong lineal pattern of staining was located in the basement membrane zone of the peripheral cornea (FIG. 4D). Nevertheless, the staining was diffuse in the superficial stroma of the limbus (FIG. 4E). Staining to laminin 5 was negative in the basement membrane zone of the entire region (FIG. 4F), suggesting total digestion of this protein during the 18-hour incubation.

Characterization of the Basement Membrane-Adhesion Complex after Epithelial Sheet Isolation.

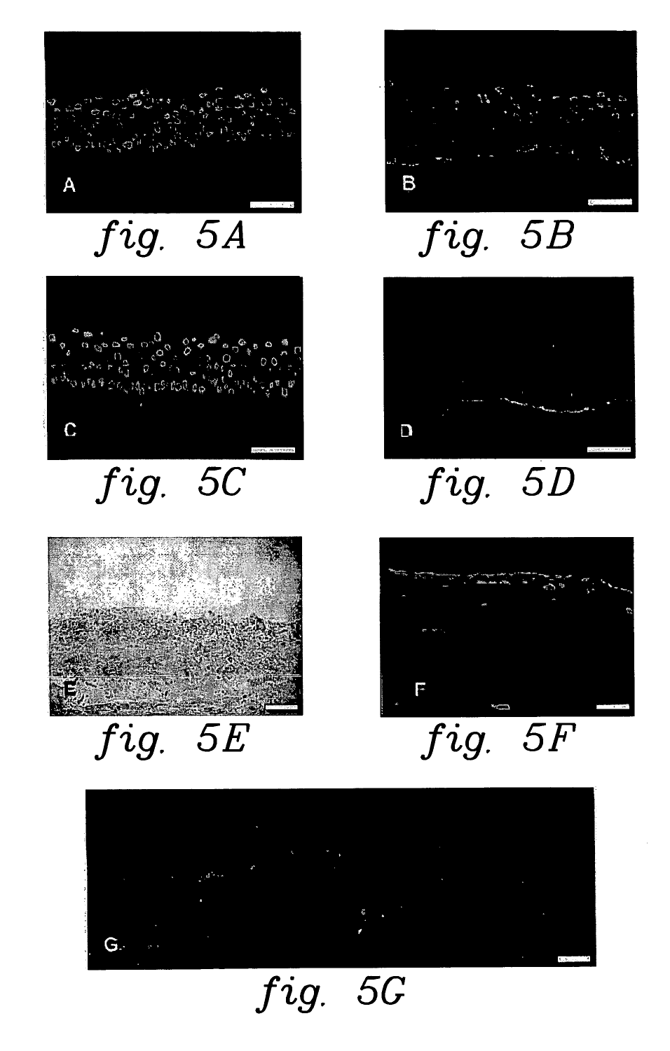

After digestion, we isolated limbal epithelial sheets and then analyzed the sheet and the remaining stroma separately by immunostaining. The staining to integrin β4 was linearly positive on the basal epithelial cell surface (FIG. 5A), but was absent on the remaining stroma (not shown). The staining to laminin 5 was negative on the entire epithelial sheet (FIG. 5B) and negative on the remaining stroma (not shown). The staining to collagen IV was sporadically positive on the basal surface of the isolated sheet (FIG. 5B), but was strongly positive in a lineal pattern on the superficial surface of the remaining stroma (FIG. 5F). Staining to collagen VII was negative on the isolated limbal sheet (FIG. 5D), but was diffusely positive in the superficial stroma of the stromal remnant (FIG. 5G). These findings were consistent with those described in FIG. 4.

Characterization of Epithelial Outgrowth Derived from Isolated Limbal Epithelial Sheets in Culture

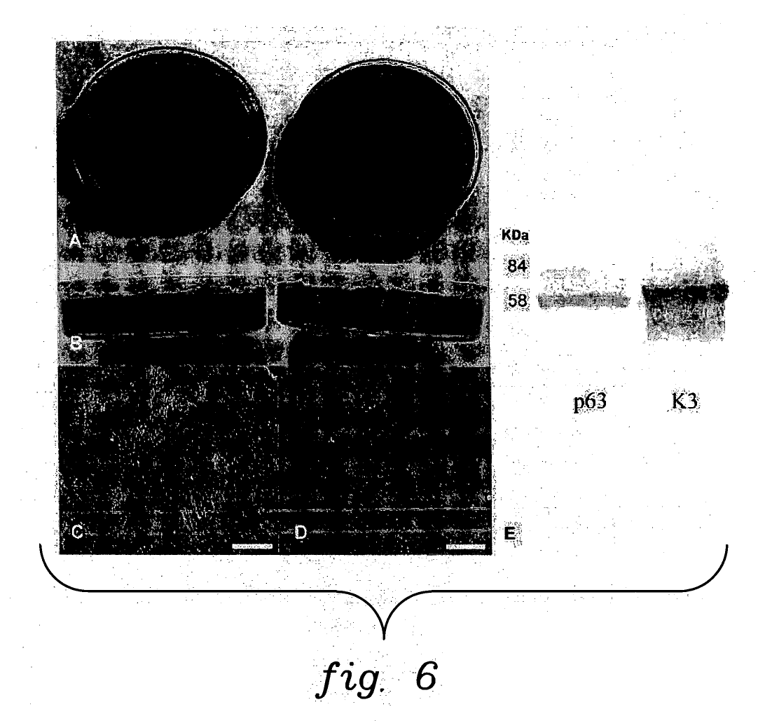

One small segment of isolated limbal epithelial sheet in a size of 1.5 mm of arch length was seeded on the center of each 60 mm plastic dish, and cultured in SHEM. Cells rapidly grew out of the sheet and reached the border of the dish in 17.7±3 days (FIG. 6A). Epithelial cells continued to grow onto the sidewall of the dish to the level where the medium was (FIG. 6B). Phase contrast microscopy showed that cells appeared to be small in size and formed a compact monolayer (FIGS. 6C and 6D). Western blot analysis of proteins extracted from these cells on confluency showed a positive band of p63 at 60 kDa and a positive band of keratin 3 at 64 kDa (FIG. 6E).

### EXAMPLE 3 (

A Reproducible Method of Expanding Human Keatocytes on Amniotic Membrane and Characterization of Results.)

Materials And Methods:

Human keratocytes were isolated from central corneal buttons by digestion in 1 mg/ml of collagenase A in DMEM and seeded on plastic or the stromal matrix of human amniotic membrane (AM) in DMEM with different concentrations of FBS. Upon confluency, cells on AM were continuously subcultured for 6 passages on AM or plastic. In parallel, cells cultured on plastic at passages 3 and 11 were seeded back to AM. Cell morphology and intercellular contacts were assessed by phase contrast microscopy and LIVE AND DEATH assay, respectively. Expression of keratocan was determined by RT-PCR and Western blotting.

Results:

Trephined stroma yielded 91,600±26,300 cells (ranging from 67,000 to 128,000 cells per corneal button). Twenty-four hours after seeding, cells appeared dendritic on AM but fibroblastic on plastic even in 10% FBS. Such a difference in morphology correlated with expression of keratocan assessed by RT-PCR and Western blot, which was high and continued at least to passage 6 on AM even in 10% FBS, but was rapidly lost each time when cells on AM were passaged on plastic. Fibroblasts continuously cultured on plastic to passage 3 and 11 did not revert their morphology or synthesize keratocan when re-seeded on plastic in 1% FBS or on AM.

Conclusion:

Human keratocytes maintain their characteristic morphology and keratocan expression when subcultured on AM stromal matrix even in the presence of high serum concentrations. This method can be used to engineer a new corneal stroma.

### EXAMPLE 4

(Isolation and Culture of Human Keratocytes on Plastic or AM Membranes; Analyses)

Materials:

The tissue culture plastic plates (six-well) and 30 mm culture dishes were from Becton Dickinson (Lincoln Park, N.J.). Culture plate inserts used for fastening AM were from Millipore (Berford, Mass.). Amphotericin B, Dulbecco's modified Eagle's medium (DMEM), fetal bovine serum (FBS), gentamicin, Hank's balanced salt solutions (HBSS), HEPES-buffer, phosphate buffered saline (PBS), 0.05% trypsin/0.53mM EDTA, and TRIZOL® reagent were purchased from Gibco-BRL (Grand Island, N.Y.). 4-15% gradient SDS-polyacrylamide gel and horseradish secondary anti-rabbit antibody were from Biorad (Hercules, Calif.). Collagenase A was obtained from Roche (Indianapolis, Ind.). Aminobenzamidine, EDTA tetrasodium salt, guanidine, hydrochloric acid, isopropanolol, chloroform, endo-β-galactosidase, sodium acetate and urea were from Sigma (St. Louis, Mo.). A LIVE AND DEATH Assay® was obtained from Molecular Probes (Eugene, Oreg.).

Methods:

Isolation of Human Keratocytes.

Human corneas stored in humid chambers for less than 4 days were obtained from the Florida Lions Eye Bank (Miami, Fla.). An 8 mm Barron's trephine was used to remove a central corneal button. After scrapping off the corneal epithelium with a cell scraper and peeling off Descemet's membrane, the remaining corneal stroma was cut into 0.5 mm×0.5 mm pieces. These stromal pieces (˜12 per cornea) were then incubated at 37° C. for 45 min in DMEM containing 1 mg/ml collagenase A in a plastic dish. After incubation, collagenase A was removed by pipetting and the digested stromal pieces were incubated in a second aliquot of collagenase A for another 45 min or until the tissue became “smeared” onto the bottom of the dish. The digested tissue was then centrifuged at 800×g for 5 min and resuspended in 1.5 ml of DMEM containing 20 mM HEPES, 50 μg/ml gentamicin and 1.25 μg/ml amphotericin per cornea. This keratocytes-containing cell suspension was then seeded on plastic dishes or the stromal side of the AM.

Primary Culture of Keratocytes on Plastic or Amniotic Membrane.

Human AM preserved according to the method described by Lee and Tseng (See Lee S-H, Tseng S C G, “Amniotic membrane transplantation for persistent epithelial defects with ulceration,” Am J Ophthalmol, 1997;123:303-12.) was kindly provided by Bio-Tissue (Miami, Fla.). After thawing, human AM was incubated in HBSS containing 0.1% EDTA for 30 min at 37° C., and the amniotic epithelium was then denuded using an AMOILS® epithelial scrubber (Innova, Toronto, Ontario, Canada). Epithelially denuded AM with the stromal side facing up was tightened to a small plastic insert −32 mm diameter—using a rubber band in a manner similar to what has been reported. See Grueterich M, Espana E, Tseng S C, “Connexin 43 expression and proliferation of human limbal epithelium on intact and denuded amniotic membrane,” Invest Ophthalmol Vis Sci 2002;43:63-71. The keratocyte cell suspension prepared from one corneal button was seeded on each 32 mm insert or a 35 mm plastic dish. They were cultured in a medium containing DMEM supplemented with 10% FBS, and the medium was changed every 2-3 days. In a separate experiment, cultures grown in DMEM containing 10% FBS for 24 h were switched to DMEM containing 10%, 5%, or 1% FBS and cultured for 10 days.

Subculture of Keratocytes on Plastic and Amniotic Membrane

When the primary culture on AM reached 70-80% confluence, cells were dissociated into single cells by incubation in HBSS containing 0.05% trypsin and 0.53 mM EDTA at 37° C. for 20 min, followed by vigorous pipetting. After centrifuging at 800×g for 5 min, cells were re-suspended in DMEM containing 10% FBS, subdivided into 2 equal parts, with one being seeded onto AM stroma and the other on a plastic dish. They were cultured in DMEM containing 10% FBS. The AM culture was subcultured to either AM or plastic culture in the same manner as described above for a total of 6 passages. In parallel, cells grown on plastic in DMEM containing 10% FBS were continuously subcultured at 1:3 split on plastic. Cells on plastic at passage 3 and 11 were seeded on plastic in DMEM containing 1%, 5%, or 10% FBS or on AM stromal matrix in DMEM containing 10% FBS to see if there was any reversibility in morphology and keratocan expression.

Morphological Analysis Using LIVE-DEATH ASSAYS®

At each passage on AM or plastic, cell morphology was documented by phase-contrast microscopy, and in some instances analyzed by the staining with LIVE-DEATH ASSAY® according to a methods described by Poole et al. (See Poole C A, et al., “Keratocyte networks visualized in the living cornea using vital dyes,” J Cell Sci 1993; 106:685-92.), and the manufacturer. Briefly, after the removal of the culture medium, cells were washed twice with HBSS and incubated for 40 min with 0.5 ml LIVE-DEATH ASSAY® consisting of 2 mM calcein-AM, and 4 mM ethidium homodimer in PBS. After washing with PBS, cells were examined by a NikonTe-2000u Eclipse epi-fluorescent microscope (Nikon, Tokyo, Japan).

Reverse Transcription-Polymerase Chain Reaction (RT-PCR).

Total RNA was extracted by TRIZOL® reagent from two 8 mm central corneal buttons, which had been minced with a blade and sonicated at 6000 rpm using a TISSUE TEAROR™ sonicator (Biospec Products INC, Racine, Wis.) as a positive control. Total RNA was similarly extracted from cells cultured on plastic or AM. Total RNA equivalent to 1×10⁵ cultured cells or one corneal button was subjected to RT-PCR based on a protocol recommended by Promega. The final concentration of RT reaction was 10 mM Tris-HCl (pH 9.0 at 25° C.), 5 mM MgCl₂, 50 mM KCl, 0.1 % Triton X-100, 1 mM each dNTP, 1 unit/ml recombinant RNase in ribonucleases inhibitor, 15 units AMV reverse transcriptase, 0.5 mg Oligo(dT)15 primer and total RNA in a total volume of 20 ml. The reaction was kept at 42° C. for 60 min. One tenth sample from RT was used for subsequent PCR with the final concentration of PCR reaction being 10 mM Tris-HCl (pH 8.3 at 25° C.), 50 mM KCl, 1.5 mM Mg(OAc)2, 1.25 units of Taq DNA polymerase in a total volume of 50 ml using primers shown in Table 1. The PCR mixture was first denatured at 94° C. for 5 min then amplified for 30 cycles (94° C., 1 min; 60° C., 1 min; 72° C., 1 min) using PTC-100 Programmable thermal Controller (MJ Research Inc, USA). After amplification, 15 ml of each PCR product and 3 ml of 6× loading buffer were mixed and electrophoresed on a 1.5% agarose gel in 0.5× Tris-boric acid-EDTA (TBE) containing 0.5 mg/ml ethidium bromide. Gels were photographed and scanned.

TABLE 1
RT-PCR Primer Sequences

| | Sequence | | | |
|---|---|---|---|---|
| | 5′-3′ | cDNA | Size of | |
| Primer | AccessionNumber | Location | PCR product | GenBank |
| Keratocan | | | | |
| Sense | ATGGCAGGCACAATCTGTTTCATC | (1-24) | 1059 bp | NM_007035 |
| Antisense | TTAAATAATGACAGCCTGCAGAAG | (1059-1036) | | |
| Lumican | | | | |
| Sense | ACCATGAGTCTAAGTGCATTT | (1-18) | 1015 bp | NM_002345 |
| Antisense | CAATTAAGAGTGACTTCGU | (1015-997) | | |
| Collagen | | | | |
| type III-a1 | | | | |
| Sense | TCTTTGAATCCTAGCCCATCTG | (4816-4837) | 568 bp | NM_000090 |
| Antisense | TGTGACAAAAGCAGCCCCATAA | (5385-5361) | | |
| GAPDH | | | | |
| Sense | GAAGGTGAAGGTCGGAGTCAACG | (60-82) | 573 bp | BC029618 |
| Antisense | GCGGCCATCACGCCACAGTTTC | (654-633) | | |

Western Blot Analysis

Total cellular protein was extracted from cells cultured on AM and plastic at P2 and P4 using TRIZOL® Reagent for total RNA (see total RNA isolation). After complete removal of the aqueous phase which containing total RNA and precipitation of DNA with ethanol, the protein in the phenol-ethanol supernatant was precipitated with isopropyl alcohol. The protein pellet was then extracted in 4 M guanidine-HCl containing 10 mM sodium acetate, 10 mM sodium EDTA, 5 mM aminobenzamidine, and 0.1 M ε-amino-n-caproic acid at 4° C. overnight. The extracts were dialyzed exhaustively in distilled water and the water insoluble fraction was dissolved in 0.1 M Tris-acetate solution (pH 6.0) containing 6 M urea. The protein concentration was measured by spectrophotometer at OD 280 nm. One hundred μg protein aliquots were incubated with endo-β-galactosidase (0.1 U/ml, Sigma) at 37□ C overnight. Equal volume of 2×SDS sample buffer was added into samples, boiled for 5 min, electrophoresed on an SDS-PAGE gradient (4-15%) gel, and transferred to a nitrocellulose membrane. These membranes were pre-incubated with blocking buffer and probed with an affinity-purified polyclonal antibody raised against a synthetic peptide (RSVRQVYEVHDSDDWTIH) corresponding to 18 N-terminal amino acids of the predicted human keratocan protein (a gift from Dr. Albert de la Chapelle, Ohio State University). See Pellegata N S, et al., “Mutations in KERA, encoding keratocan, cause cornea plana,” Nat Genet 2000;25:91-5. Immunoreactivity was visualized with an enhanced chemiluscent reagent (Perkin Elmer, Boston, Mass., USA). Two normal human corneas were minced with a blade and sonicated at 6000 rpm using a Tissue Tearor™ sonicator for use as a positive control. Proteins extraction was carried out using the same procedure described for expanded cells but avoiding TRIZOL®.

Results:

Morphological Differences in Primary Cultures

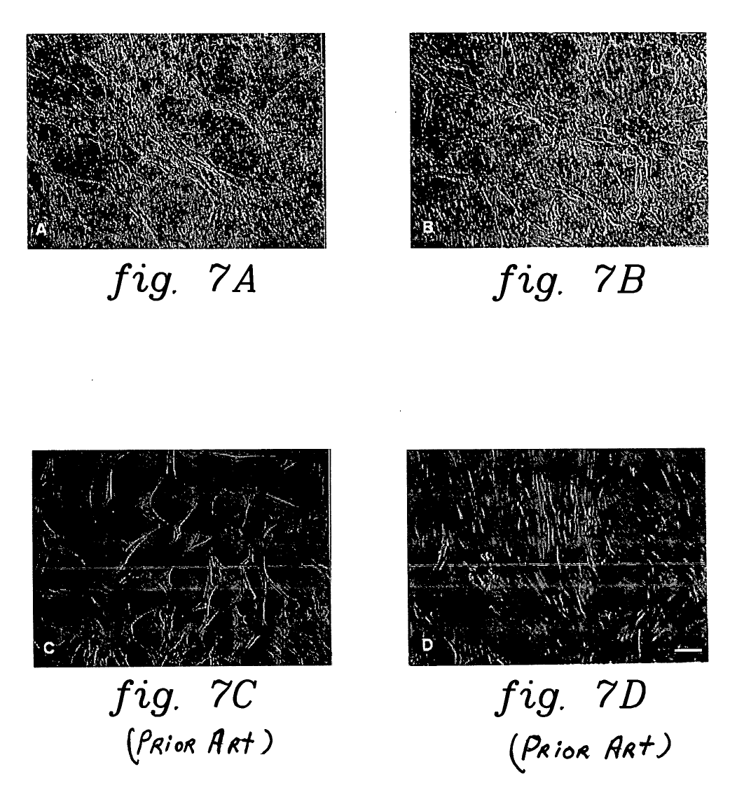

Cell suspension obtained after collagenase digestion yielded 91,600±26,300 cells (ranging from 67000 to 128000 cells per corneal button). Within 24 h after seeding, cells attached to plastic and AM matrix and exhibited a distinctly different morphology. On AM stroma, cells were dendritic or stellate in shape and formed a connecting network when grown in the presence of 1% or 10% FBS for one week (FIGS. 7A and 7B, respectively). Cells on AM matrix projected their dendritic processes in a 3-dimensional pattern. In contrast, cells on plastic dishes were evenly distributed on a flat surface and adopted a mixture of spindle and stellate shapes when cultured in 1% FBS for one week (FIG. 7C), but appeared uniformly spindle when cultured in 10% FBS (FIG. 7D, one week after seeding). Cells showed continuous proliferation with increasing concentrations of serum. In 10% FBS, cells on plastic reached confluence in 6 days and cells on AM did so in 14-17 days.

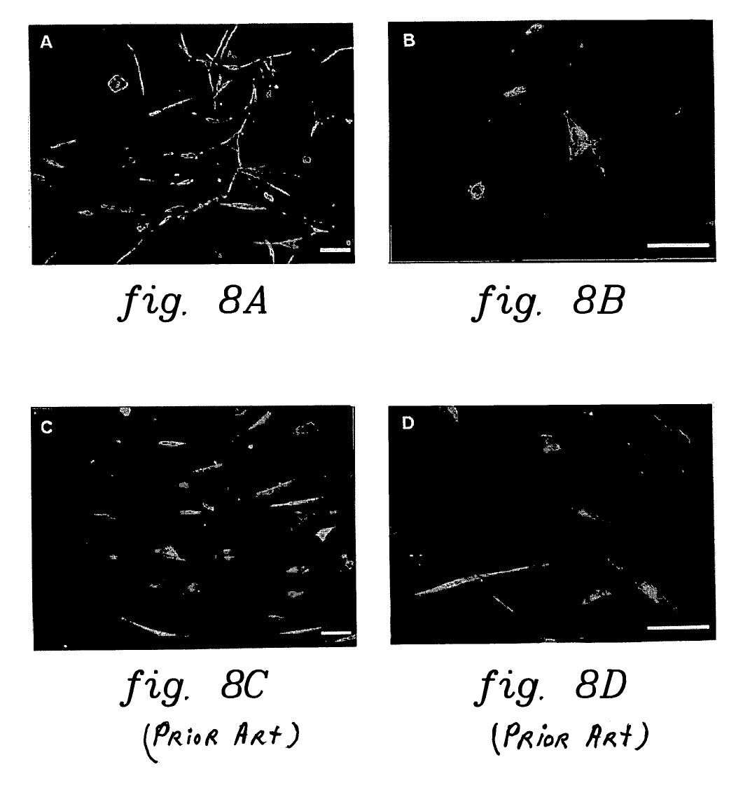

To display better the above difference in cellular morphology of these two culture systems, we used LIVE-DEATH ASSAY® to fully demarcate the entire cytoplasm. Indeed, the majority of cells grown on AM stroma in 10% FBS had a triangular-shaped cell body, and their cytoplasm was stretched into many thin dendritic processes (FIG. 8A). These processes formed extensive intercellular contacts in a three dimensional pattern (FIG. 8B). In contrast, cells grown on plastic in 10% FBS maintained spindle shaped cytoplasm with no intercellular contact (FIGS. 8C and 8D).

Morphological Differences in Continuous Passages.

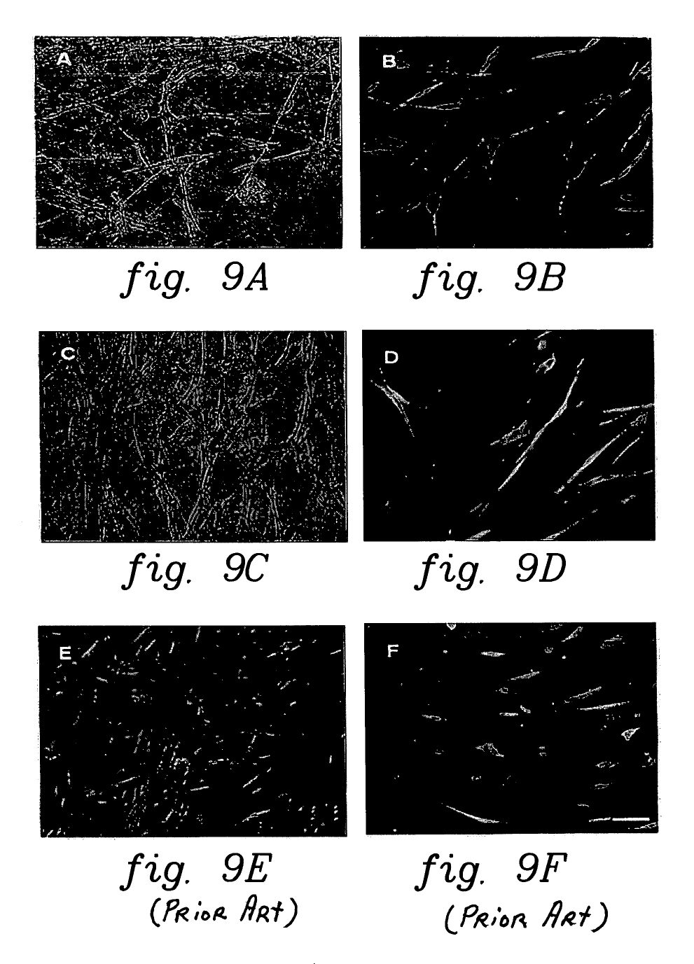

Cells continued to maintain a dendritic morphology with widespread intercellular contacts when continuously passaged from the primary culture so long as they were grown on AM stromal matrix. As shown in FIG. 9, such a dendritic morphology was maintained up to passage 2 and 4 (FIGS. 9A and 9C, respectively). Similarly, extensive intercellular contacts were also maintained when illustrated by staining with LIVE-DEATH ASSAY® (FIGS. 9B and 9D, respectively). In contrast, cells immediately adopted a spindle shape within 24 h when subcultured from the primary AM culture to a plastic dish (FIG. 9E) with a marked loss of intercellular contacts (FIG. 9F). Such a dramatic change in cell morphology from dendritic to spindle was consistently observed each time when cells on an AM culture were subsequently cultured on a plastic dish for a total of 6 passages tested so far (not shown).

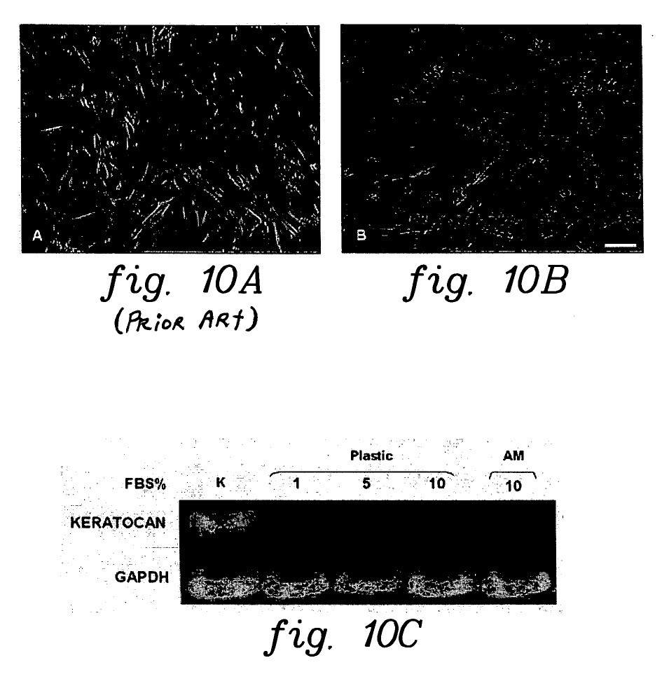

When we passaged cells that had continuously been cultured on plastic up to passage 3 to AM stromal matrix, we noted that the fibroblastic morphology (FIG. 10A) remained spindle and did not revert to a dendritic morphology (FIG. 10B). Even if they were cultured on plastic with 1% FBS, such a spindle shape was not changed (not shown). The same result was obtained when we used cells continuously cultured on plastic up to passage 10 (not shown). FIG. 10C shows that Expression of keratocan transcript (1,059 bp) by RT-PCR was found in the normal corneal stroma (K), but was not detected in cells on plastic with 1%, 5%, or 10% FBS or on AM with 10% FBS. The expression of GAPDH (573 bp) serves as a loading control.

Keratocan Expression

Reverse Transcription-Polymerase Chain Reaction (RT-PCR):

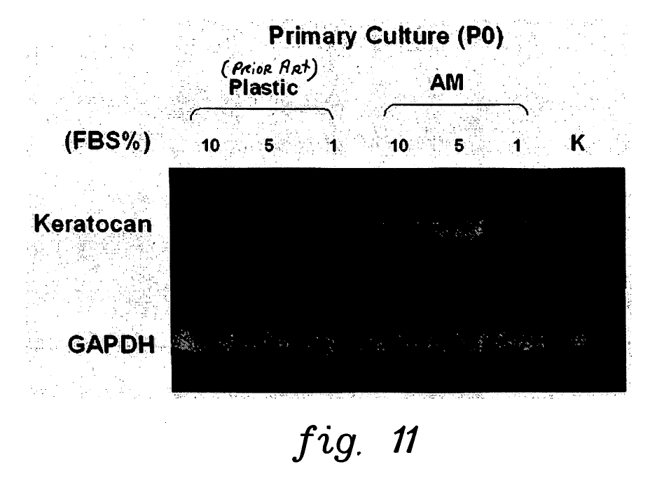

Total RNA was extracted from cells seeded on plastic and AM, and RT-PCR was used to determine the expression of keratocan transcript, which was at the size of 1059 base pair (bp). In primary cultures, cells grown on plastic barely expressed keratocan transcript in 1% FBS, but rapidly lost keratocan expression in 5% or 10% FBS (FIG. 11). In contrast, cells expressed abundant amounts of keratocan transcript in 1%, 5% and 10% FBS, with the highest noted in 5% FBS (FIG. 11).

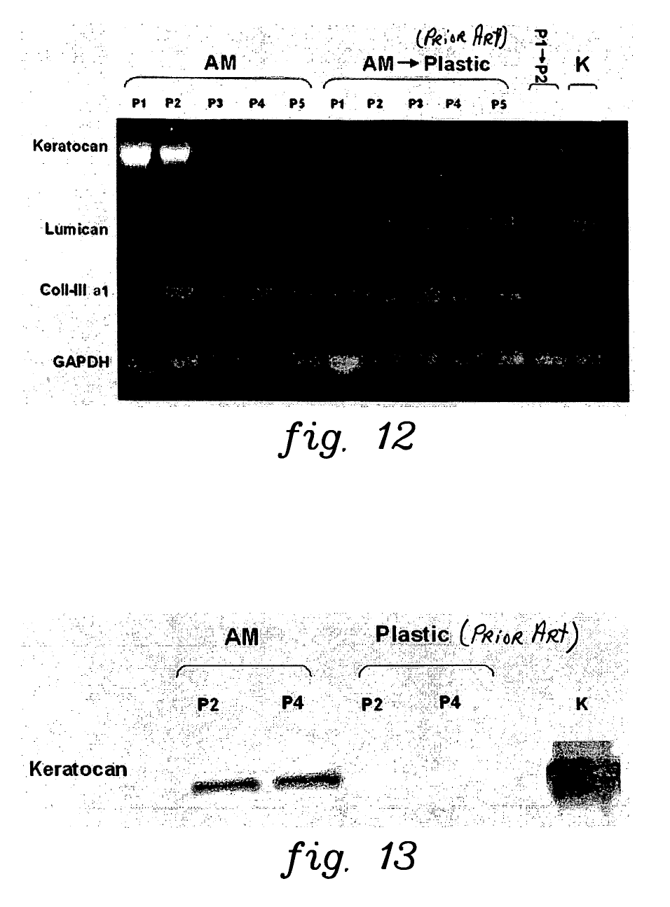

To determine whether such a difference in keratocan expression correlated with the morphological changes noted above, we continued to subculture primary culture of cells on AM for a total of 6 passages. Total RNA was extracted from AM and plastic cultures for up to passage 5. For each passage, cells cultured on AM were equally divided and subcultured on either AM or plastic. All cells were grown in DMEM containing 10% FBS. As shown in FIG. 12, using glyceraldehyde-3-phosphate dehydrogenase (GAPDH) (with a size of 573 bp) as a loading control, we noted that cells sub-cultured on plastic (Prior Art) showed reduced expression of keratocan transcript at passages 1 and 2 but did not express keratocan transcript thereafter up to passage 5. In contrast, cells subcultured on AM expressed abundant amounts of keratocan transcript at passages 1 and 2, and continued to do so up to passage 5. Such a dramatic difference was still maintained at passage 6, the last passage tested so far (not shown).

It should be noted in FIG. 12 that, compared to GAPDH (573 bp) as a loading control, keratocan transcript (1,059 bp) was expressed in all AM cultures but largely lost when subcultured from AM to plastic cultures (Prior Art), especially after passage 3. In contrast, expression of lumican transcript (1,015 bp) and collagen III-a1 transcript (568bp) was detected in both AM and (Prior art) plastic cultures. As expected, normal corneal stroma (K) expressed keratocan and lumican but not collagen 111-a1.

Unlike the aforementioned expression pattern of keratocan, transcripts of lumican (1015 base pair (bp)) and collagen III-a1 (568 bp) were uniformly expressed by cells grown on AM and plastic for up to passage 5 (FIG. 12). As a control, the normal cornea stroma (K) expressed keratocan, lumican but not collagen III-a1 (FIG. 12). The finding that collagen III-a1 is not expressed by normal corneal stroma, but expressed in wounded cornea has been reported.28

Cells continuously cultured on plastic with 10% FBS up to passage 3 did not express any keratocan transcript when subcultured on plastic even in 1% FBS, or seeded back on AM (FIG. 10). The same result was obtained for cells continuously cultured on plastic for up to passage 11 (not shown).

Western Blot Analysis

To correlate the above transcript expression with the protein expression, we performed Western blot analysis. Proteins extracted by guanidine HCl from cells grown on AM and plastic at passage 2 and 4 clearly expressed a positive band of 50 kDa, which was consistent with keratocan27 expressed by normal corneal stroma as a positive control (FIG. 13). In contrast, this protein band was not detected in proteins extracted from cells cultured on plastic at passage 2 and 4 (FIG. 13).

Literature Cited. It is believed that surgeons, scientists, and researchers will benefit from the information provided in the following papers.
1. Kenyon K R, Tseng S C G, Limbal autograft transplantation for ocular surface disorders. Ophthalmology. 1989;96:709-723.
2. Tsai R J F, Sun T-T, Tseng S C G. Comparison of limbal and conjunctival autograft transplantation for corneal surface reconstruction in rabbits. Ophthalmology. 1990;97:446-455.
3. Geggel H S, Gipson I K. Removal of viable sheets of conjunctival epithelium with dispase II. Invest Ophthalmol Vis Sci. 1985;26:15-22.
4. Khodadoust A A. Techniques for the preparation of sheets of pure corneal epithelium. Am J Ophthalmol. 1967;63 :942-945.
5. Gipson I K, Grill S M. A technique for obtaining sheets of intact rabbit corneal epithelium. Invest Ophthalmol Vis Sci. 1982;23:269-273.
6. Spurr S J, Gipson I K. Isolation of corneal epithelium with Dispase II or EDTA. Effects on the basement membrane zone. Invest Ophthalmol Vis Sci. 1985;26:818-827.
7. Gipson I K. The epithelial basement membrane zone of the limbus. Eye. 1989;3 (Pt 2):132-140.
8. Tseng S C G, Zhang S-H. Limbal epithelium is more resistant to 5-fluorouracil toxicity than corneal epithelium. Cornea. 1995;14:394-401.
9. Grueterich M, Espana E, Tseng S C. Connexin 43 expression and proliferation of human limbal epithelium on intact and denuded amniotic membrane. Invest Ophthalmol Vis Sci. 2002;43:63-71.
10. Schermer A, Galvin S, Sun T-T. Differentiation-related expression of a major 64K corneal keratin in vivo and in culture suggests limbal location of corneal epithelial stem cells. J Cell Biol. 1986; 103:49-62.
11. Pellegrini G, Dellambra E, Golisano O, Martinelli E, Fantozzi I, Bondanza S, Ponzin D, McKeon F, De Luca M. p63 identifies keratinocyte stem cells. Proc Natl Acad Sci USA. 2001;98:3156-3161.
12. Matic M, Petrov I N, Chen S, Wang C, Dimitrijevich S D, Wolosin J M. Stem cells of the corneal epithelium lack connexins and metabolite transfer capacity. Differentiation. 1997;61:251-260.
13. Wolosin J M, Xiong X, Schütte M, Stegman Z, Tieng A. Stem cells and differentiation stages in the limbo-corneal epithelium. Prog Retinal & Eye Res. 2000;19:223-255.
14. Green H, Kehinde O, Thomas J. Growth of cultured human epidermal cells into multiple epithelia suitable for grafting. Proc Natl Acad Sci USA. 1979;76:5665-5668.
15. Stenn K S, Link R, Moellmann G, Madri J, Kuklinska E. Dispase, a neutral protease from Bacillus polymyxa, is a powerful fibronectinase and type IV collagenase. J Invest Dermatol. 1989;93 :287-290.
16. Ljubimov A V, Burgeson R E, Butkowski R J, Michael A F, Sun T-T, Kenney M C. Human corneal basement membrane heterogeneitiy: topographical differences in the expression of type IV collagen and laminin isoforms. Lab Invest. 1995;72:461-473.
17. Pfefer B A. Improved methodology for cell culture of human and monkey retinal pigment epithelium. Prog Retin Eye Res. 1991;10:251-291.
18. Poumay Y, Roland I H, Leclercq-Smekens M, Leloup R. Basal detachment of the epidermis using dispase: tissue spatial organization and fate of integrin alpha 6 beta 4 and hemidesmosomes. J Invest Dermatol. 1994;102:111-117.
19. Espana E M, Grueterich M, Mateo A, Romano A, Yee S, Yee R W, Tseng S C G. Cleavage of corneal basement membrane components by ethanol exposure like LASEK. J Cat Refract Surg. 2003; in press.
20. Poole C A, Brookes N H, Clover, G M, “Keratocyte networks visualized in the living cornea using vital dyes. J Cell Sci 1993; 106:685-92.
21. Ueda A, Nishida T, Otori T, Fujita H. Electron-microscopic studies on the presence of gap junctions between corneal fibroblasts in rabbits. Cell Tissue Res 1987;249:473-5.
22. Petridou S, Masur S K. Immunodetection of connexins and cadherins in corneal fibroblasts and myofibroblasts. Invest Ophthalmol Vis Sci 1996;37:1740-8.
23. Birk D E, Lande M A, Fernandez-Madrid F R. Collagen and glycosaminoglycan synthesis in aging human keratocyte cultures. Exp Eye Res 1981;32:331-9.
24. Cintron C, Hong B S. Heterogeneity of collagens in rabbit cornea: type VI collagen. Invest Ophthalmol Vis Sci 1988;29:760-6.
25. Funderburgh J L, Conrad G W. Isoforms of corneal keratan sulfate proteoglycan. J Biol Chem 1990;265:8297-303.
26. Funderburgh J L, Funderburgh M L, Mann M M, Conrad G W. Unique glycosylation of three keratan sulfate proteoglycan isoforms. J Biol Chem 1991;266:14226-31.
27. Funderburgh J L, Funderburgh M L, Brown S J, et al. Sequence and structural implications of a bovine corneal keratan sulfate proteoglycan core protein. Protein 37B represents bovine lumican and proteins 37A and 25 are unique. J Biol Chem 1993;268:11874-80.
28. Liu C-Y, Siraishi A, Kao C W-C, et al. The cloning of mouse keratocan cDNA and genomic DNA and the characterization of its expression during eye development. J Biol Chem 1999;273:22584-8.
29. Ujita M, Shinomura T, Kimata K. Molecular cloning of the mouse osteoglycin-encoding gene. Gene 1995;158:237-40.
30. Ying S, Shiraishi A, Kao C W, et al. Characterization and expression of the mouse lumican gene. J Biol Chem 1997;272:30306-13.
31. Chakravarti S, Magnuson T, Lass J H, et al. Lumican regulates collagen fibril assembly: skin fragility and corneal opacity in the absence of lumican. J Cell Biol 1998;141:1277-86.
32. Chakravarti S, Petroll W M, Hassell J R, et al. Corneal opacity in Lumican-null mice: defects in collagen fibril structure and packing in the posterior stroma. Invest Ophthalmol Vis Sci 2000;41:3365-73.
33. Beals M P, Funderburgh J L, Jester J V, Hassell J R. Proteoglycan synthesis by bovine keratocytes and corneal fibroblasts: Maintenance of the keratocyte phenotype in culture. Invest Ophthalmol Vis Sci 1999;40:1658-63.
34. Jester J V, Barry-Lane P A, Cavanagh H D, Petroll W M. Induction of a-smooth muscle actin expression and myofibroblast transformation in cultured corneal keratocytes. Cornea 1996;15:505-16.
35. Masur S K, Cheung J K H, Antohi S. Identification of integrins in cultured corneal fibroblasts and in isolated keratocytes. Invest Ophthalmol Vis Sci 1993;34:2690-8.
36. Desmouliére A, Geinoz A, Gabbiani F, Gabbiani G. Transforming growth factor β-1 induces a-smooth muscle actin expression in granulation tissue myofibroblasts and in quiescent and growing cultured fibroblasts. J Cell Biol 1993;122:103-11.
37. Masur S K, Dewal H S, Dinh T T, et al. Myofibroblasts differentiate from fibroblasts when plated at low density. Proc Natl Acad Sci USA 1996;93:4219-23.
38. Gabbiani G, Chaponnier C, Hüttner I. Cytoplasmic filaments and gap junctions in epithelial cells and myofibroblasts during wound healing. J Cell Biol 1978;76:561-8.
39. Dahl I M, Johnsen W, Anseth A, Prydz H. The synthesis of glycosaminoglycans by corneal stroma cells in culture. Exp Cell Res 1974;88:193-7.
40. Dahl I M, Coster L. Proteoglycan biosynthesis in cultures of corneas and corneal stroma cells from adult rabbits. Exp Eye Res 1978;27:175-90.
41. Berryhill B L, Beales M P, Hassell J R. Production of prostaglandin D synthase as a keratan sulfate proteoglycan by cultured bovine keratocytes. Invest Ophthalmol Vis Sci 2001;42:1201-7.
42. Berryhill B L, Kader R, Kane B, et al. Partial restoration of the keratocyte phenotype to bovine keratocytes made fibroblastic by serum. Invest Ophthalmol Vis Sci 2002;43:3416-21.
43. Jester J V, Petroll W M, Cavanagh H D. Corneal stromal wound healing in refractive surgery: the role of myofibroblasts. Prog Retin Eye Res 1999;18:311-56.
44. Lee S-H, Tseng S C G. Amniotic membrane transplantation for persistent epithelial defects with ulceration. Am J Ophthalmol 1997;123:303-12.
45. Grueterich M, Espana E, Tseng S C. Connexin 43 expression and proliferation of human limbal epithelium on intact and denuded amniotic membrane. Invest Ophthalmol Vis Sci 2002;43:63-71.
46. Pellegata N S, Dieguez-Lucena J L, Joensuu T, et al. Mutations in KERA, encoding keratocan, cause cornea plana. Nat Genet 2000;25:91-5.
47. White J, Werkmeister J A, Ramshaw J A, Birk D E. Organization of fibrillar collagen in the human and bovine cornea: collagen types V and III. Connect Tissue Res 1997;36:165-74.
48. Tseng S C G, Li D-Q, Ma X. Suppression of Transforming Growth Factor isoforms, TGF-b receptor II, and myofibroblast differentiation in cultured human corneal and limbal fibroblasts by amniotic membrane matrix. J Cell Physiol 1999; 179:325-35.
49. Choi T H, Tseng S C G. In vivo demonstration of suppression of scarring and autologous blood-induced inflammation by intrastromal implantation of human amniotic membrane in rabbit corneas. Invest. Ophthalmol Vis. Sci. 40, S328. 1999.
50. Lee S-B, Li D-Q, Tan D T H, et al. Suppression of TGF-β signaling in both normal conjunctival fibroblasts and pterygial body fibroblasts by amniotic membrane. Curr Eye Res 2000;20:325-34.
51. Choi T H, Tseng S C G. In vivo and in vitro demonstration of epithelial cell-induced myofibroblast differentiation of keratocytes and an inhibitory effect by amniotic membrane. Cornea 2001;20:197-204.
52. Solomon A, Rosenblatt M, Monroy D C, et al. Suppression of Interleukin-1a and Interleukin-1b in the human corneal epithelial cells cultured on the amniotic membrane matrix. Br J Ophthalmol 2001;85:444-9.
53. Tseng S C G, Tsubota K. Amniotic Membrane Transplantation for Ocular Surface Reconstruction. In: Holland E J, Mannis M J, eds. Ocular Surface Disease, 1st ed. NY, Berlin, Heidelberg: Springer, 2002; chap. 20.
   Equivalents

While this invention has been particularly shown and described with references to preferred embodiments thereof, it will be understood by those skilled in the art that various changes in form and details may be made therein without departing from the scope of the invention encompassed by the appended claims.

1. A method of enzymatically isolating a substantially intact, viable limbal epithelial sheet comprising the steps of:
a) obtaining limbus from a biopsy of a donor eye chosen from an eye of a living individual, an eye of a living, related individual, and a cadaveric eye, the limbus comprising limbal epithelium and an underlying stroma;
b) contacting the limbus with a solution comprising Dispase 2, for a period of time and under conditions sufficient to loosen a limbal epithelial sheet from the stroma, thereby forming a loosely adherent limbal epithelial sheet; and
c) mechanically separating the loose epithelial sheet from the underlying stroma, thereby isolating a substantially intact, viable, limbal epithelial sheet.

2. The method of claim 1, wherein the solution contacting the limbus according to step b) further comprises a substance chosen from SHEM, a polyhydroxy alcohol, a sugar, and combinations thereof.

3. The method of claim 2, wherein the polyhydroxy alcohol used is chosen from sorbitol, mannitol, and galactitol.

4. The method of claim 1, wherein the conditions of step b) include maintaining a temperature from about 0° C. to about 37° C. for at least half an hour.

5. A surgical graft comprising an isolated, substantially intact, viable, limbal epithelial sheet, the limbal epithelial sheet prepared by a process comprising the steps of:
a) obtaining limbus from a biopsy of a donor eye of an individual or a cadaveric eye, the limbus comprising limbal epithelium and an underlying stroma;
b) contacting the limbus with a solution comprising Dispase 2 for a period of time and under conditions sufficient to loosen a limbal epithelial sheet from the stroma, thereby forming a loosely adherent limbal epithelial sheet; and
c) mechanically separating the loose epithelial sheet from the underlying stroma, thereby forming a surgical graft comprising an isolated, substantially intact, viable, limbal epithelial sheet.

6. The surgical graft of claim 5, wherein the solution contacting the limbus according to step b) further comprises a substance chosen from SHEM, a polyhydroxy alcohol, a sugar, and combinations thereof.

7. A surgical graft comprising an isolated, substantially intact, viable, limbal epithelial sheet.

8. The surgical graft of claim 7, wherein the limbal epithelial sheet retains at least one stem cell characteristic.

9. The surgical graft of claim 7, wherein the limbal epithelial sheet comprises cells that do not express keratin 3, or connexin 43, but may express p63.

10. The surgical graft of claim 7, wherein the graft is an allograft.

11. The surgical graft of claim 7, wherein the graft is an autograft.

12. The surgical graft of claim 7, further comprising amniotic membrane.

13. The surgical graft of claim 12, further comprising mesenchymal cells.

14. The surgical graft of claim 13, wherein the mesenchymal cells are chosen from fetal mesenchymal cells, keratocytes, fibroblasts, endothelial cells, melanocytes, cartilage cells, bone cells, hematopoietic stem cells, bone marrow mesenchymal stem cells, adult mesenchymal stem cells, and combinations thereof.

15. The surgical graft of claim 12, further comprising keratocytes.

16. The surgical graft of claim 12, wherein the amniotic membrane has cells and an extracellular matrix; and wherein prior to using the amniotic membrane as a surgical graft the cells of the amniotic membrane have been killed while maintaining the integrity of the extracellular matrix.

17. The surgical graft of claim 12, wherein the amniotic membrane has been freeze-dried prior to using the amniotic membrane as a surgical graft.

18. A method of expanding ex vivo epithelial stem cells present in a limbal epithelial sheet, comprising the steps of:
a) enzymatically isolating a limbal epithelial sheet according to the method of claim 1;
b) contacting the limbal epithelial sheet with a basement membrane side of an amniotic membrane, thereby forming a composite comprising limbal epithelial sheet and amniotic membrane; and
c) culturing the composite for a period of time and under conditions sufficient to enable the epithelial stem cells to expand.

19. A surgical graft comprising limbal epithelial cells expanded according to the method of claim 18.

20. A surgical graft comprising keratocytes on amniotic membrane.

21. The surgical graft of claim 20, wherein the amniotic membrane has cells and an extracellular matrix; and wherein prior to using the amniotic membrane as a surgical graft the cells of the amniotic membrane have been killed while maintaining the integrity of the extracellular matrix.

22. The surgical graft of claim 20, wherein the amniotic membrane has been freeze-dried prior to using the amniotic membrane as a surgical graft.

23. The surgical graft of claim 20, wherein the graft is an allograft.

24. The surgical graft of claim 20, wherein the graft is an autograft.

25. A method of expanding mesenchymal cells ex vivo, while maintaining the phenotype of the mesenchymal cells, comprising:
a) contacting a stromal side of an anmiotic membrane with at least one type of mesenchymal cells, thereby forming a composite comprising the mesenchymal cells and the amniotic membrane; and
b) culturing the composite in a serum-containing medium for a period of time and under conditions sufficient to enable the mesenchymal cells to expand while maintaining the phenotype of the mesenchymal cells.

26. A surgical graft comprising mesenchymal cells expanded ex vivo according to the method of claim 25.

27. The method of claim 25, wherein the mesenchymal cells allowed to expand are keratocytes, and the keratocytes maintain their phenotype.

28. The method of claim 27, wherein the phenotype maintained by the keratocytes includes at least one of dendritic morphology and keratocan expression.

29. A surgical graft comprising human keratocytes expanded ex vivo according to the method of claim 27.

30. The surgical graft of claim 19 comprising mesenchymal cells chosen from fetal mesenchymal cells, keratocytes, fibroblasts, endothelial cells, melanocytes, cartilage cells, bone cells, hematopoietic stem cells, bone marrow mesenchymal stem cells, adult mesenchymal stem cells, and combinations thereof.

31. The surgical graft of claim 26, wherein the mesenchymal cells are chosen from fetal mesenchymal cells, keratocytes, fibroblasts, endothelial cells, melanocytes, cartilage cells, bone cells, hematopoietic stem cells, bone marrow mesenchymal stem cells, adult mesenchymal stem cells, and combinations thereof.


SEQUENCE LISTING (10 sequences)

SEQ ID NO: 1 - DNA, 24 bp, Artificial Sequence
  Description of Artificial Sequence Primer
atggcaggcacaatctgtttcatc

SEQ ID NO: 2 - DNA, 24 bp, Artificial Sequence
  Description of Artificial Sequence Primer
gaagacgtccgacagtaataaatt

SEQ ID NO: 3 - DNA, 21 bp, Artificial Sequence
  Description of Artificial Sequence Primer
accatgagtctaagtgcattt

SEQ ID NO: 4 - DNA, 20 bp, Artificial Sequence
  Description of Artificial Sequence Primer
ttgcttcagtgagaattaac

SEQ ID NO: 5 - DNA, 22 bp, Artificial Sequence
  Description of Artificial Sequence Primer
tctttgaatcctagcccatctg

SEQ ID NO: 6 - DNA, 22 bp, Artificial Sequence
  Description of Artificial Sequence Primer
aataccccgacgaaaacagtgt

SEQ ID NO: 7 - DNA, 23 bp, Artificial Sequence
  Description of Artificial Sequence Primer
gaaggtgaaggtcggagtcaacg

SEQ ID NO: 8 - DNA, 22 bp, Artificial Sequence
  Description of Artificial Sequence Primer
ctttgacaccgcactaccggcg

SEQ ID NO: 9 - DNA, 15 bp, Artificial Sequence
  Description of Artificial Sequence Primer
ttttttttttttttt

SEQ ID NO: 10 - protein, 18 aa, Artificial Sequence
  Description of Artificial Sequence Synthetic peptide
RSVRQVYEVHDSDDWTIH